(19) United States
(12) Patent Application Publication
(10) Pub. No.: US 2012/0053534 A1
(43) Pub. Date: Mar. 1, 2012

(54) INTRAVAGINAL DEVICES COMPRISING ANTICHOLINERGIC AGENTS, AND METHODS OF MAKING THEREOF

(76) Inventors: Anu Mahashabde, Kendall Park, NJ (US); Jiaxiang Tsao, Nanuet, NY (US)

(21) Appl. No.: 13/166,309

(22) Filed: Jun. 22, 2011

Related U.S. Application Data
(60) Provisional application No. 61/357,325, filed on Jun. 22, 2010.

(51) Int. Cl. A61M 31/00; B28B 1/14

(52) U.S. Cl. 604/285; 264/241

(57) ABSTRACT

The present invention is directed to an intravaginal device comprising: (a) an annular first matrix comprising a pocket and a pocket wall, wherein the pocket wall has a uniform thickness, and wherein the pocket wall encompasses the pocket; and (b) a second matrix comprising an anticholinergic agent, wherein the second matrix is located in the pocket.

The present invention is also directed to a method of making an intravaginal device, the method comprising: (a) placing a first matrix into a mold, the mold being shaped so as to form an annular intravaginal device comprising a pocket and a pocket wall, wherein the pocket wall has a uniform thickness, and wherein the pocket wall encompasses the pocket; (b) curing the first matrix; (c) placing a second matrix comprising an anticholinergic agent in the pocket; and (d) curing the second matrix.

This application claims the benefit of the filing date of U.S. Appl. No. 61/357,325, filed Jun. 22, 2010, the entirety of which is fully incorporated by reference herein.

### FIELD OF THE INVENTION

The present invention relates to intravaginal devices comprising: (a) an annular first matrix comprising a pocket and a pocket wall, wherein the pocket wall has a uniform thickness, and wherein the pocket wall encompasses the pocket; and (b) a second matrix comprising an anticholinergic agent located in the pocket.

The present invention also relates to methods of making intravaginal devices, the methods comprising: (a) placing a first matrix into a mold, the mold being shaped so as to form an annular intravaginal device comprising a pocket and a pocket wall, wherein the pocket wall has a uniform thickness, and wherein the pocket wall encompasses the pocket; (b) curing the first matrix; (c) placing a second matrix comprising an anticholinergic agent in the pocket; and (d) curing the second matrix.

### BACKGROUND OF THE INVENTION

Overactive bladder (“OAB”) affects millions of individuals worldwide, a majority of those being women. In individuals with OAB, the detrusor muscle that controls the voluntary relaxation of the bladder during urination contracts spontaneously and involuntarily leading to a variety of symptoms such as urinary incontinence, urinary urgency, and increased urinary frequency.

Currently, OAB is treated by administration of the anticholinergic agent oxybutynin, Oxybutynin is believed to affect the detrusor muscle, leading to relaxation of the bladder and subsequent reduction of spontaneous involuntary contractions.

Currently marketed modes of oxybutynin administration include both oral (syrup or tablets), marketed under the tradenames DITROPAN® (syrup and tablets, Ortho-McNeil-Janssen Pharmaceutical, Inc., Titusville, N.J.) and LYRINEL XL® (tablets, Janssen-Cilag EMEA, Beerse, Belgium), and transdermal patches, marketed under the tradename OXYTROL® (Watson Pharmaceutical, Inc., Morristown, N.J.). Deleterious side effects can occur upon oral and transdermal administration of oxybutynin, e.g., dry eyes, dizziness, blurred vision, constipation, and/or headaches.

### BRIEF SUMMARY OF THE INVENTION

The present invention is directed to an intravaginal device comprising: (a) an annular first matrix comprising a pocket and a pocket wall, wherein the pocket wall has a uniform thickness, and wherein the pocket wall encompasses the pocket; and (b) a second matrix comprising an anticholinergic agent, wherein the second matrix is located in the pocket.

In some embodiments, the first matrix comprises an optionally substituted polymer selected from the group consisting of polysiloxane polymers, polyalkylene polymers, polystyrene polymers, polyvinyl acetate polymers, polyvinyl chloride polymers, polyester polymers, polyurethane polymers, acrylic polymers, nylon polymers, dacron polymers, teflon polymers, and combinations thereof.

In some embodiments, the optionally substituted polymer is a polysiloxane polymer of Formula (I):

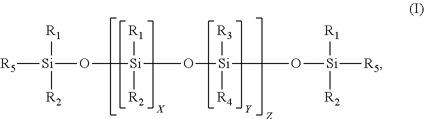

wherein X is 1 to 200; Y is 1 to 200; Z is 1 to 300; and R₁, R₂, R₃, R₄, and R₅ are independently selected from the group consisting of (C₁₋₆)alkyl, amino(C₁₋₆)alkyl, hydroxy(C₁₋₆)alkyl, haloalkyl, cyano(C₁₋₆)alkyl, thio(C₁₋₆)alkyl, carboxy(C₁₋₆)alkyl, aryl(C₁₋₆)alkyl, (C₁₋₆)alkoxy(C₁₋₆)alkyl, (C₂₋₆)alkenyl, amino(C₃₋₁₀)alkenyl, hydroxy(C₃₋₁₀)alkenyl, halo(C₂₋₆)alkenyl, cyano(C₂₋₆)alkenyl, thio(C₃₋₁₀)alkenyl, carboxy(C₃₋₁₀)alkenyl, aryl(C₂₋₆)alkenyl, (C₂₋₆)alkynyl, (C₁₋₆)heteroalkyl, (C₂₋₆)heteroalkenyl, (C₂₋₆)heteroalkynyl, (C₁₋₆)alkoxy, (C₃₋₁₀)alkenyloxy, (C₁₋₆)alkylenedioxy, amino(C₂₋₆)alkoxy, hydroxy(C₂₋₆)alkoxy, halo(C₁₋₆)alkoxy, cyano(C₁₋₆)alkoxy, thio(C₁₋₆)alkoxy, carboxy(C₂₋₆)alkoxy, aryl(C₁₋₆)alkoxy, (C₁₋₆)alkoxy(C₂₋₆)alkoxy, halo(C₁₋₆)alkoxy(C₂₋₆)alkoxy, mono(C₁₋₆)alkylamino, di(C₁₋₆)alkylamino, (C₁₋₆)alkylcarbonylamino, (C₂₋₆)alkenylcarbonylamino, (C₆₋₁₄)arylcarbonylamino, (C₁₋₆)alkoxycarbonylamino, (C₆₋₁₀)aryloxycarbonylamino, (C₁₋₆)alkylcarbonyl, (C₂₋₆)alkenylcarbonyl, (C₆₋₁₀)arylcarbonyl, (C₁₋₆)alkoxycarbonyl, (C₆₋₁₄)aryloxycarbonyl, (C₁₋₆)alkylsulfcmylamino, (C₂₋₆)alkenylsulfonylamino, and (C₆₋₁₄)arylsulfonylamino. In some embodiments, at least one of R₁, R₂, R₃, and R₄ is a haloalkyl. In some embodiments, X is 1 to 2; Y is 1 to 2; Z is 100 to 200; R₁ is trifluoropropyl; R₂, R₃, and R₄ are independently C₁-C₃ alkyl; and R₅ is vinyl. In some embodiments, the optionally substituted polymer is 3,3,3-trifluompropyl methyldimethyl polysiloxane.

In some embodiments, the first matrix comprises 50% to 100% by weight halogenated siloxane polymer.

In some embodiments, the first matrix comprises 80% to 95% by volume of the device. In some embodiments, the first matrix comprises 80% to 95% by weight of the device.

In some embodiments, the pocket extends from 10° to 180° around the perimeter of the first matrix. In some embodiments, the pocket extends from 80° to 120° around the perimeter of the first matrix. In some embodiments, the pocket has a cross-sectional diameter of 3 mm to 8 mm. In some embodiments, the pocket wall has a uniform thickness of 1 mm to 4 mm. In some embodiments, the pocket has a volume of 0.7 cm³ to 1.5 cm³.

In some embodiments, the second matrix comprises an optionally substituted polymer selected from the group consisting of polysiloxane polymers, polyalkylene polymers, polystyrene polymers, polyvinyl acetate polymers, polyvinyl chloride polymers, polyester polymers, polyurethane polymers, acrylic polymers, nylon polymers, dacron polymers, teflon polymers, and combinations thereof. In some embodiments, the second matrix comprises a polysiloxane polymer.

In some embodiments, the second matrix comprises a polysiloxane polymer of Formula (II):

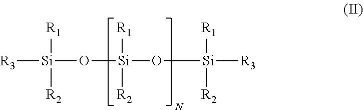

wherein R₁, R₂ and R₃ are independently selected from the group consisting of alkoxy, alkyl, alkynyl, alkynyl, alkenyl, alkylacryloyloxy, acryloyloxy, alkenylalkyl, aryl, and hydrogen; and N is 50 to 300. In some embodiments, R₁ and R₂ are independently alkyl or hydrogen.

In some embodiments, the second matrix comprises 30% to 80% by weight polysiloxane polymer.

In some embodiments, the second matrix comprises 5% to 50% by volume of the device. In some embodiments, the second matrix comprises 5% to 50% by weight of the device.

In some embodiments, the anticholinergic agent is homogenously dispersed throughout the second matrix. In some embodiments, the anticholinergic agent is selected from the group consisting of oxybutynin, tolterodine, trospium, solifenacin, darifenacin, dicyclomine, propantheline, propiverine, bethanechol, methylbenactyzium, scopolamine, and pharmaceutically acceptable salts thereof. In some embodiments, the anticholinergic agent is oxybutynin or a pharmaceutically acceptable salt thereof. In some embodiments, the anticholinergic agent comprises 20% to 70% by weight of the second matrix.

In some embodiments, the first matrix further comprises a slit, wherein the slit extends a length of the pocket.

The present invention is also directed to a method of making an intravaginal device, the method comprising: (a) placing a first matrix into a mold, the mold being shaped so as to form an annular intravaginal device comprising a pocket and a pocket wall, wherein the pocket wall has a uniform thickness, and wherein the pocket wall encompasses the pocket; (b) curing the first matrix; (c) placing a second matrix comprising an anticholinergic agent in the pocket; and (d) curing the second matrix.

In some embodiments, the mold is shaped so as to form an annular intravaginal device comprising a pocket and a pocket wall, wherein the pocket wall has a uniform thickness, wherein the pocket wall encompasses the pocket, and wherein a slit extends a length of the pocket. In some embodiments, the anticholinergic agent is homogenously dispersed in the second matrix. In some embodiments, the anticholinergic agent is selected, from the group consisting of oxybutynin, tolterodine, trospium, solifenacin, darifenacin, dicyclomine, propantheline, propiverine, bethanechol, methylbenactyzium, scopolamine, and pharmaceutically acceptable salts thereof. In some embodiments, the anticholinergic agent is oxybutynin or a pharmaceutically acceptable salt thereof.

### BRIEF DESCRIPTION OF THE FIGURES

FIG. 1 depicts a top view of an intravaginal ring having a first matrix (101) comprising a pocket (102), and a second matrix (103) located in the pocket, wherein the pocket is encompassed by a pocket wall (104). The length of the pocket around the perimeter of the first matrix is denoted by the variable (y). The pocket wall has a uniform thickness, i.e., 105a, 105b, and 105c are substantially the same length.

FIG. 2 depicts a top view of an intravaginal ring having an inner perimeter (201), an outer perimeter (202), an inner diameter (203), and outer diameter (204).

FIG. 3A depicts a side view of an intravaginal ring showing a cross-section having a first matrix (301) comprising a pocket (303) and a pocket wall (302), wherein the pocket wall has a uniform thickness, and wherein the pocket wall encompasses the pocket.

FIG. 3B depicts a side view of an intravaginal ring showing a cross-section of a vaginal ring having a first matrix (301) comprising a pocket (302) and a pocket wall (303), and a second matrix (304) comprising an anticholinergic agent located in the pocket.

FIG. 4 depicts a side view of an intravaginal ring having a first matrix (401) having a pocket (402), and a slit (403), wherein the slit extends a length of the pocket.

### DETAILED DESCRIPTION OF THE INVENTION

The present invention is directed to intravaginal devices comprising: (a) an annular first matrix comprising a pocket and a pocket wall, wherein the pocket wall has a uniform thickness, and wherein the pocket wall encompasses the pocket; and (b) a second matrix comprising an anticholinergic agent located in the pocket.

As used herein, an “intravaginal device” refers to an object suitable for placement in the vaginal tract. In some embodiments, the intravaginal device provides for administration or application of an anticholinergic agent to the vaginal and/or urogenital tract of a subject, including, e.g., the vagina, cervix, or uterus of a female. As used herein, “female” refers to any animal classified, as a mammal, including humans and non-humans, such as, but not limited to, domestic and farm animals, zoo animals, sports animals, and pets. In some embodiments, female refers to a human female. In some embodiments, the female is a menopausal woman. In some embodiments, the female is a peri-menopausal woman.

In some embodiments, the female refers to a human female, wherein the female meets one or more criteria selected from (1) predominant or pure urge incontinence consisting of ≧10 pure or predominant discrete urge incontinence episodes per week, (2) an average urinary frequency of ≧8 voids per 24 hours, and (3) an average total void volume of ≦3.0 L per 24 hours, in some embodiments, the female is a human female having all three criteria described above. In some embodiments, the female is a human menopausal or peri-menopausal woman having all three criteria described above.

The intravaginal devices of the present invention comprise an anticholinergic agent. As used herein, an “anticholinergic agent” refers to a compound that blocks the neurotransmitter acetylcholine in the central and the peripheral nervous system. Anticholinergic agents suitable for use with the present invention comprise agents that have a localized effect, as well as systemically acting anticholinergic agents that act at a point remote from the vaginal or urogenital tract. Anticholinergic agents suitable for use with the present invention include, but are not limited to, oxybutynin, tolterodine, trospium, solifenacin, darifenacin, dicyclomine, propantheline, propiverine, bethanechol, methylbenactyzium, scopolamine, combinations thereof, and pharmaceutically acceptable salts thereof.

In some embodiments, the anticholinergic agent is oxybutynin, tolterodine, trospium, solifenacin, darifenacin, dicyclomine, propantheline, propiverine, or pharmaceutically acceptable salts thereof.

In some embodiments, the anticholinergic agent is oxybutynin or a pharmaceutically acceptable salt thereof, such as, e.g., oxybutynin hydrochloride. Oxybutynin is represented by the chemical formula C₂₂H₃₁NO₃, the International Union of Pure and Applied Chemistry (IUPAC) name 4-diethylaminobut-2-ynyl2-cyclohexyl-2-hydroxy-2-phenyl-ethanoate, Chemical Abstracts Service, (CAS) number 5633-20-5, and the PubChem Compound identification number 4634. As used herein, the term “oxybutynin” refers to oxybutynin as well as its pharmaceutically acceptable salts, esters, hydrates, prodrugs, or derivatives thereof unless otherwise noted.

In some embodiments, the intravaginal devices are annular in shape. As used herein, “annular” refers to a shape of relating to, or forming a ring. Annular shapes suitable for use with the present invention include a ring, an oval, an ellipse, a toroid, and the like. In some embodiments, the intravaginal devices of the present invention are a vaginal ring.

Materials used in the intravaginal devices of the present invention can include any materials suitable for placement in the vaginal tract. In some embodiments, the materials used in the intravaginal device are nontoxic, physiologically suitable, and/or non-absorbable in a subject, i.e., they are not absorbed in the vaginal tract. The materials used in the present invention are compatible with an anticholinergic agent. Compatible materials include those materials that are inert, chemically stable, do not chemically interact with, or otherwise affect and/or alter the anticholinergic agent. In some embodiments, the materials are pliable, malleable, and/or capable of being suitably shaped for intravaginal administration.

The intravaginal devices of the present invention can be flexible. As used herein, “flexible” refers to the ability of a solid or semi-solid to bend or withstand stress and strain without being damaged or broken. For example, the device of the present invention can be deformed or flexed, such as, for example, using finger pressure (e.g., applying pressure from opposite external sides of the device using the fingers), and upon removal of the pressure, substantially return to its original shape. The flexible properties of the intravaginal device of the present invention are useful for enhancing user comfort, and also for ease of administration to the vaginal tract and/or removal of the device from the vaginal tract.

The intravaginal devices of the present invention comprise a first matrix. As used herein, a “first matrix” refers to any solid, semi-solid, or gel medium. In some embodiments, the first matrix is an amorphous polymer network formed when a polymer or a mixture of polymers undergo cross-linking. Each polymer is comprised, of monomeric units, which are linked together to form the polymer. The monomeric units can comprise carbon, hydrogen, oxygen, silicon, halogen, and combinations thereof. The first matrix can be shaped by molding, extrusion, coextrusion, compression, or combinations thereof.

In some embodiments, the first matrix is permeable to the anticholinergic agent. In some embodiments, the first matrix is permeable to oxybutynin and/or water. In some embodiments, the first matrix can be chosen due to its mechanical and physical properties (e.g., solubility or permeability of an anticholinergic agent in the material).

In some embodiments, the first matrix comprises various polymers that are compatible with the vaginal tract. In some embodiments, the first matrix comprises a polysiloxane, a polyalkylene, a polystyrene, a polyvinyl acetate, a polyvinyl chloride, a polyester, a polyurethane, an acrylic, nylon, a dacron, a teflon, or a combination thereof.

As used herein, a “polysiloxane polymer” refers to any of various compounds containing alternate silicon and oxygen atoms in either a linear or cyclic arrangement usually with one or two organic groups attached to each silicon atom. For example, polysiloxane polymers can include substituted polysiloxanes, and diorganopolysiloxanes such as diarylpolysiloxanes and dialkylpolysiloxanes.

In some embodiments, the first matrix comprises an optionally substituted polymer selected from the group consisting of polysiloxane polymers, polyalkylene polymers, polystyrene polymers, polyvinyl acetate polymers, polyvinyl chloride polymers, polyester polymers, polyurethane polymers, acrylic polymers, nylon polymers, dacron polymers, teflon polymers, and combinations thereof.

In some embodiments, the optionally substituted polymer is a polysiloxane polymer of Formula (I):

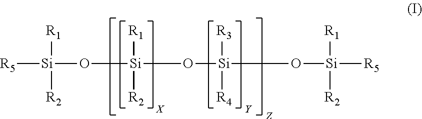

wherein X is 1 to 200; Y is 1 to 200; Z is 1 to 300; and R₁, R₂, R₃, R₄, and R₅ are independently selected from the group consisting of (C₁₋₆)alkyl, amino(C₁₋₆)alkyl, hydroxy(C₁₋₆)alkyl, haloalkyl, cyano(C₁₋₆)alkyl, thio(C₁₋₆)alkyl, carboxy(C₁₋₆)alkyl, aryl(C₁₋₆)alkyl, (C₁₋₆)alkoxy(C₁₋₆)alkyl, (C₂₋₆)alkenyl, amino(C₃₋₁₀)alkenyl, hydroxy(C₃₋₁₀)alkenyl, halo(C₂₋₆)alkenyl, cyano(C₂₋₆)alkenyl, thio(C₃₋₁₀)alkenyl, carboxy(C₃₋₁₀)alkenyl, aryl(C₂₋₆)alkenyl, (C₂₋₆)alkynyl, (C₁₋₆, heteroalkyl, (C₂₋₆)heteroalkenyl, (C₂₋₆)heteroalkynyl, (C₁₋₆)alkoxy, (C₃₋₁₀)alkenyloxy, (C₁₋₆)alkylenedioxy, amino(C₂₋₆)alkoxy, hydroxy(C₂₋₆)alkoxy, halo(C₁₋₆)alkoxy, cyano(C₁₋₆)alkoxy, thio(C₁₋₆)alkoxy, carboxy(C₂₋₆)alkoxy, aryl(C₁₋₆)alkoxy, (C₁₋₆)alkoxy(C₂₋₆)alkoxy, halo(C₁₋₆)alkoxy(C₂₋₆)alkoxy, mono(C₁₋₆)alkylamino, di(C₁₋₆)alkylamino, (C₁₋₆)alkylcarbonylamino, (C₂₋₆)alkenylcarbonylamino, (C₆₋₁₄)arylcarbonylamino, (C₁₋₆)alkoxycarbonylamino, (C₆₋₁₀)aryloxycarbonylamino, (C₁₋₆)alkylcarbonyl, (C₂₋₆)alkenylcarbonyl, (C₆₋₁₀)arylcarbonyl, (C₁₋₆)alkoxycarbonyl, (C₆₋₁₄)aryloxycarbonyl, (C₁₋₆)alkylsulfonylamino, (C₂₋₆)alkenylsulfonylamino, and (C₆₋₁₄)arylsulfonylamino. In some embodiments, at least one of R₁, R₂, R₃, and R₄ is a haloalkyl.

In some embodiments, the first matrix is a halogenated siloxane polymer, wherein at least one of R₁, R₂, R₃, and R₄ is a mono-haloalkyl, di-haloalkyl, or tri-haloalkyl. In some embodiments, the haloalkyl is a bromoalkyl, chloroalkyl, fluoroalkyl, or iodoalkyl. In some embodiments, the haloalkyl is a trifluoroalkyl. In some embodiments, the haloalkyl is a trifluoroethyl, trifluoropropyl, or trifluorobutyl In some embodiments, the haloalkyl is a difluoroethyl, difluoropropyl, or difluorobutyl.

In some embodiments. X is 1 to 90, 10 to 80, or 20 to 70. In some embodiments, X is 1 to 10, 1 to 5, or 1 to 3. In some embodiments, Y is 1 to 90, 10 to 80, or 20 to 70. In some embodiments, Y is 1 to 10, 1 to 5, or 1 to 3. In some embodiments, Z is 10 to 250, 50 to 200, or 75 to 150. As one of skill in the art would recognize, the values of X and Y can vary in each Z subunit. Thus, e.g., X is 3 and Y is 4 in a first Z subunit, and X is 10 and Y is 2 in a second Z subunit.

In some embodiments, R₁ is a trifluoropropyl; R₂, R₃, and R₄ are independently C₁-C₃ alkyl; R₅ is vinyl; X is 1 to 2; Y is 1 to 2; and Z is 100 to 200.

In some embodiments, the first matrix comprises 3,3,3-trifluoropropyl methyldimethyl polysiloxane, e.g., the trifluoropropylmethyl polymer sold by NuSil Technology (Carpinteria, Calif.).

Throughout the disclosure, all expressions of percentage, ratio, and the like are “by weight” unless otherwise indicated. As used herein, “by weight” is synonymous with the term “by mass,” and indicates that a ratio or percentage defined herein is according to weight rather than volume, thickness, or some other measure.

In some embodiments, the first matrix is 50% to 1.00% by weight halogenated siloxane polymer. In some embodiments, the first matrix is 75% to 95% by weight halogenated siloxane polymer. In some embodiments, the first matrix is 80% to 90% by weight halogenated siloxane polymer.

In some embodiments, the first matrix is 80% to 95% by weight of the intravaginal device. In some embodiments, the first matrix is 80% to 95% by volume of the intravaginal device.

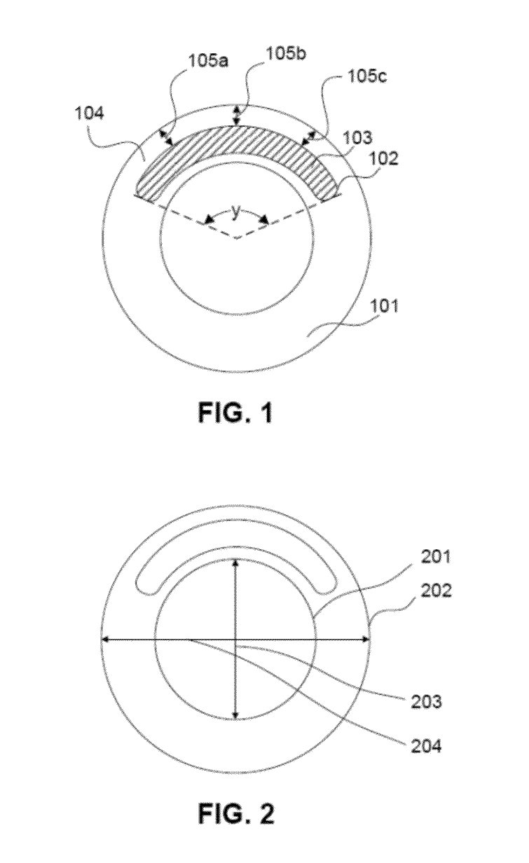

The first matrix comprises a pocket and a pocket wall, wherein the pocket wall has a uniform thickness, and wherein the pocket wall encompasses the pocket. As used herein, “pocket” refers to an indentation, groove, furrow, cut, impression, notch, recess, or likewise depression along the surface of the first matrix, which is encompassed by a pocket wall, and wherein the pocket wall has a uniform thickness. See, e.g., FIGS. 1, 2, 3A, and 3B. In some embodiments, a “pocket” as defined herein can be exposed to the exterior of the device via a slit which extends a length of the pocket. Thus, the term “pocket” does not include a bore or other type of cavity that extends any length through the device, since (a) a bore contains at least one distinct entrance from the surface into the first matrix, and (b) a bore does not have a pocket wall of uniform thickness. In some embodiments a pocket of the present invention can be beneficial since anticholinergic agents in a second matrix can be released without having to pass through a separate matrix, e.g., the first matrix.

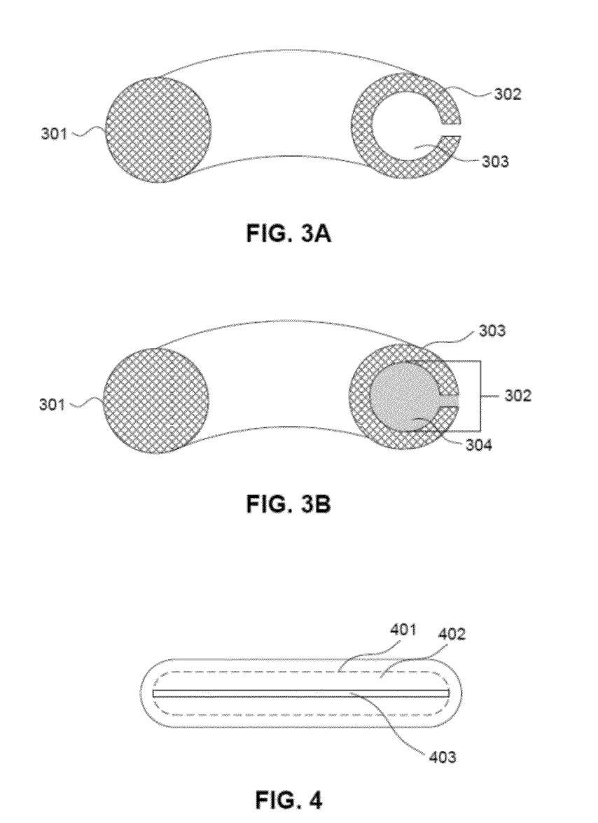

As used herein, “pocket wall” refers to a portion of the first matrix that defines the lateral boundaries of the pocket. See, e.g., FIGS. 3A and 3E. Thus, the volume defined by the pocket wall comprises the pocket. The pocket wall has a uniform thickness, wherein the distance from the pocket to the lateral outer surface of the device is the same. In some embodiments, the pocket wall has a uniform thickness of 0.5 mm to 5 mm. In some embodiments, the pocket wall has a uniform thickness of 1 mm to 4 mm. In some embodiments, the pocket wall has a uniform thickness of 1.5 mm to 3 mm. In some embodiments, the pocket wall has a uniform thickness of 1 mm to 2 mm. A pocket wall of uniform thickness can allow the anticholinergic agent in the second matrix to be uniformly released from the intravaginal device through the pocket wall.

As used herein, “encompass” or “encompasses the pocket” refers to the degree by which the pocket wall covers the lateral surface area of the pocket. Thus, the pocket wall encompasses the pocket when the pocket wall covers 95% or more of the lateral surface area of the pocket. In some embodiments, the pocket wall encompasses the pocket when the pocket wall covers 90% or more of the lateral surface area of the pocket. In some embodiments, the pocket wall encompasses the pocket when the pocket wall covers 85% or more of the lateral surface area of the pocket. In some embodiments, the pocket wall encompasses the pocket when the pocket wall covers 80% or more of the lateral surface area of the pocket. By way of example, in some embodiments, the pocket can be tubular in shape, wherein 95% or more of the lateral surface area of the tubular pocket comprises the pocket wall.

In some embodiments, the length of the pocket can vary. For example, in some embodiments, the first matrix is annular in shape and the pocket of the first matrix can extend around a portion of the entire perimeter of the annular matrix. See, e.g., FIG. 1. In some embodiments the pocket extends from 10° to 180° around the perimeter of the first matrix. In some embodiments, the pocket extends from 80° to 120° around the perimeter of the first matrix. In some embodiments, the pocket extends 180°, 150°, 120°, 100°, 90°, 80°, 70°, 60°, 45°, 30°, or 10° around the perimeter of the annular first matrix. These variables are represented by the variable “y” in FIG. 1. In some embodiments, the pocket has a cross-sectional diameter of 3 mm to 8 mm, 4 mm to 7 mm, or 5 mm to 6 mm. In some embodiments, the pocket has a total volume of 7 cm³ to 15 cm³, 8 cm³ to 14 cm³, 9 cm³ to 13 cm³, or 10 cm³ to 12 cm³. In some embodiments, the first matrix comprises one or more pockets, e.g., two, three, four, or five pockets.

In some embodiments, the first matrix further comprises a slit on the outer perimeter of the first matrix, wherein the slit extends a length of the pocket. As used herein “slit” refers to any narrow opening, incision, fissure, aperture, breach, cleavage, crack, crevice, gash, split, chasm, or cut in the outer perimeter of the first matrix. In some embodiments, the slit has a uniform width. In some embodiments, the width of the slit is 0.1 mm to 2 mm. In some embodiments, the width of the slit is 0.2 mm to 1 mm. In some embodiments, the width of the slit is 0.4 mm to 0.6 mm. In some embodiments, the width of the slit is 0.5 mm. A slit extending a length of the pocket can allow for a uniform release of active agent from the device without having to pass through a separate matrix, e.g., the first matrix.

The intravaginal devices of the present invention further comprise a second matrix. As used herein, “second matrix” refers to any solid, semi-solid, or gel medium. In some embodiments, the second matrix is an amorphous polymer network formed when a polymer or a mixture of polymers undergo cross-linking. Each polymer is comprised of monomeric units, which are linked together to form the polymer. The monomeric units can comprise carbon, hydrogen, oxygen, silicon, halogen, or a combination thereof. The second matrix can be shaped by flow, molding, or extrusion. In some embodiments, the second matrix can be flexible. In some embodiments, the second matrix can be chosen due to its mechanical and physical properties (e.g., solubility of an anticholinergic agent in the material). In some embodiments, the second matrix is placed within the pocket of the first matrix as a liquid or gel (i.e., a low viscosity state) and the second matrix is polymerized, cured, or solidified.

In some embodiments, the devices comprise more than two matrices, e.g., three or four matrices. In some embodiments, when two or more matrices are present, an anticholinergic agent is in each matrix, or optionally in only one matrix.

In some embodiments, the anticholingeric agent can be homogeneously dispersed in the second matrix. As used herein, “homogeneous” refers to a matrix that has a substantially uniform distribution of the anticholinergic agent throughout the matrix. In some embodiments, the anticholinergic is present in a uniform concentration throughout the second matrix.

In some embodiments, the anticholinergic agent is heterogeneously dispersed in the second matrix. As used herein, “heterogeneous” refers to a matrix that does not have a substantially uniform distribution of the anticholinergic agent throughout the matrix. For example, there can be segments, regions, or areas of the matrix with varying amounts of the anticholinergic agent located throughout the matrix.

In some embodiments, the second matrix comprises the same material as the first matrix. In some embodiments, the second matrix comprises a different material than that of the first matrix. For example, in some embodiments, the second matrix comprises a siloxane polymer and the first matrix comprises a halogenated siloxane polymer. In some embodiments, the siloxane polymer comprises a polymer of Formula II,

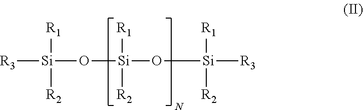

wherein R₁, R₂, and R₃ are independently selected from the group consisting of alkoxy, alkyl, alkynyl, alkynyl, alkenyl, alkylacryloyloxy, acryloyloxy, alkenylalkyl, aryl, and hydrogen; and N is 50 to 300. In some embodiments, R₁ and R₂ are independently alkyl or hydrogen. As one of skill in the art can appreciate, in a single polymer chain, the R₁ and/or R₂ substituents can vary. For example, in a single polymer chain, the R₁ and R₂ substituents can include various different alkyl substituents, e.g., methyl, ethyl, propyl, butyl, and the like.

The amount of the anticholinergic agent in the intravaginal device can vary. For example, in some embodiments, the second matrix comprises 20% to 70% by weight anticholingeric agent. In some embodiments, the second matrix comprises 30% to 60% by weight anticholingeric agent. In some embodiments, the second matrix comprises 40% to 50% by weight anticholingeric agent. In some embodiments, the second matrix comprises 50% by weight anticholingeric agent.

The amount of oxybutynin or a pharmaceutically acceptable salt thereof in the intravaginal device can vary. For example, in some embodiments, the second matrix comprises 20% to 70% by weight oxybutynin or a pharmaceutically acceptable salt thereof. In some embodiments, the second matrix comprises 30% to 60% by weight oxybutynin or a pharmaceutically acceptable salt thereof. In some embodiments, the second matrix comprises 40% to 50% by weight oxybutynin or a pharmaceutically acceptable salt thereof. In some embodiments, the second matrix comprises 50% by weight oxybutynin or a pharmaceutically acceptable salt thereof.

In some embodiments, the second matrix is 30% to 80% by weight siloxane polymer. In some embodiments, the second matrix is 40% to 70% by weight siloxane polymer. In some embodiments, the second matrix is 50% to 60% by weight siloxane polymer.

In some embodiments, the second matrix is 5% to 50% by volume of the device. In some embodiments, the second matrix is 5% to 25%, 8% to 20%, 10% to 18%, or 12% to 15% by volume of the device.

In some embodiments, the second matrix is 5% to 50% by weight of the device. In some embodiments, the second matrix is 5% to 25%, 8% to 20%, 10% to 18%, or 12% to 15% by weight of the device.

The device of the present invention is of any size suitable for placement in a vaginal tract of the subject for which it is administered. In some embodiments, the device of the present invention has a cross-sectional diameter of 1 mm to 10 mm. As used herein, a “cross-sectional diameter” refers to the longest straight line segment that passes through the center of a cross-section of the intravaginal device. See, e.g., FIG. 3A. In some embodiments, the device has a cross-sectional diameter of 1 mm to 10 mm, 2 mm to 9 mm, 3 mm to 7 mm, 4 mm to 6.5 mm, 5 mm to 6 mm, or 6 mm.

In some embodiments, the device of the invention has an outer diameter of 40 mm to 80 mm. As used herein, an “outer diameter” refers to any straight line segment that passes through the center of the device, the center being viewed from a top view of the intravaginal device, and whose endpoints are each on the outer perimeter of the device. See e.g., FIG. 2 (204). In some embodiments, the device has an outer diameter of 40 mm to 80 mm, 45 mm to 65 mm, or 50 mm to 60 mm.

In some embodiments, the device of the invention has an inner diameter of 10 mm to 60 mm. As used herein, an “inner diameter” refers to any straight line segment that passes through the center of the device, the center being viewed from a top view of the intravaginal device, and whose endpoints are on the inner perimeter of the device. See, e.g., FIG. 2 (203). In some embodiments, the device has an inner diameter of 10 mm to 60 mm, 10 mm to 50 mm, 10 mm to 40 mm, 20 mm to 40 mm, 10 mm to 30 mm, or 10 mm to 20 mm.

In some embodiments, the intravaginal device of the present invention further comprises an excipient. Where two or more matrices are present in the device, an excipient is present in each matrix, or optionally in only one matrix, i.e., in either the first or the second matrix. As used herein, an “excipient” refers to a substance that is used in the formulation of the intravaginal device of the present invention, and, by itself, generally has little or no therapeutic value. One of skill in the art will recognize that a wide variety of pharmaceutically acceptable excipients is used including those listed in the Handbook of Pharmaceutical Excipients, Pharmaceutical Press 4th Ed. (2003) and Remington: The Science and Practice of Pharmacy, Lippincott Williams & Wilkins, 21st Ed. (2005), which are incorporated herein by reference in their entirety. As used herein, the term “pharmaceutically acceptable” refers to those compounds, materials, and/or compositions which are, within the scope of sound medical judgment, suitable for contact with the tissues of human beings and animals without excessive toxicity, irritation, allergic response, or other possible complications commensurate with a reasonable benefit/risk ratio. In some embodiments, the excipient can enhance permeabilization of the matrix and the release rate of the anticholinergic agent from the intravaginal vaginal ring. Examples of such excipients include, but are not limited to, a saturated polyglycolyzed glyceride, a block copolymer surfactant, an emulsifier, glyceryl monolaurate, microcrystalline cellulose, hydroxyethylcellulose, ethylcellulose, hydroxypropyl methylcellulose, polymethylmethacrylate, polyvinylpyrollidone, and mixtures thereof. The intravaginal device of the invention can also include excipients that enhance and/or promote absorption of the anticholinergic agent across the vaginal mucosa. Absorption promoters include but are not limited to nonionic surface active agents, bile salts, organic solvents, interesterified stone oil, and ethoxydiglycol. Other excipients, such as water, saline, additives, fillers, or other pharmaceutically acceptable and/or therapeutically effective compounds, can also be added to the device of the present invention.

In some embodiments, the present invention is also directed to a method of making an intravaginal device, the method comprising: (a) placing a first matrix into a mold, the mold being shaped so as to form an intravaginal device comprising a pocket and a pocket wall, wherein the pocket wall has a uniform thickness, and wherein the pocket wall encompasses the pocket; (b) curing the first matrix; (c) placing a second matrix comprising an anticholinergic agent in the pocket; and (d) curing the second matrix.

In some embodiments, the method of the present invention further comprises curing the first matrix, the second matrix, and/or all of the matrices of the intravaginal device. As used herein, “curing” refers to a process useful to solidify, harden, or cross-link a substantially homogeneous composition of the present invention. Curing can comprise heating, drying, cooling, crystallizing, cross-linking, photo-curing (e.g., exposing to monochromatic or broad-band ultraviolet, visible, or infrared light), or combinations thereof. In some embodiments, the matrix can be cured at 0° to 200° C. In other embodiments, the matrix is cured at 120° C. to 180° C., or 150° C. In some embodiments, the matrix is cured at room temperature. In some embodiments, the matrix is cured in a mold press. In some embodiments

The present invention is also directed to an intravaginal device made by the method of the present invention. Various methods can be used to make the intravaginal devices of the present invention. Various means of producing intravaginal devices are known in the art. See, e.g., U.S. Pat. Nos. 6,544,546; 6,394,094; and 4,155,991 of which the disclosure of each is incorporated herein by reference.

In some embodiments, compression molding is used to form the device of the present invention. Compression molding generally involves compressing a substantially homogeneous mixture to form a compressed matrix and can be achieved by, e.g., the use of a die press. As used herein, “compressed” refers to a mixture that has been compacted or fused under pressure. A compressed mixture has a density that is greater than the mixture prior to compression.

In some embodiments, the matrix is in a heated liquid state prior to being placed in the mold. The heated liquid matrix can then solidify upon cooling. In some embodiments, the matrix in a liquid state solidifies with the addition of a catalyst.

In some embodiments, the intravaginal device of the present invention is a flexible, opaque, or molded silicone product with a cross-sectional diameter of 9 mm to 10 mm and an outer diameter of 55 mm to 60 mm. In some embodiments, the intravaginal device is an intravaginal ring having a pocket having a cross-sectional diameter of 4 mm to 6 mm.

In some embodiments, the pocket of the oxybutynin intravaginal ring can be filled with a paste-like mixture comprising 50% to 60% silicone and 40% to 50% oxybutynin. In some embodiments, the silicone/oxybutynin mixture can cured into a solid, achieving the shape and form of the pocket.

The present invention is also directed to an intravaginal device for administering an anticholinergic agent, the device comprising: (a) an annular first matrix comprising a pocket and a pocket wall, wherein the pocket wall has a uniform thickness, and wherein the pocket wall encompasses the pocket; and (b) a second matrix comprising an anticholinergic agent located, in the pocket.

In some embodiments, the anticholinergic agent is released from the intravaginal device at a rate of 0.1 mg/day to 20 mg/day. As used herein, the “rate of release” or “release rate” refers to an amount of anticholinergic agent that is released from the intravaginal device over a defined period of time. In other embodiments, the anticholinergic agent is released from the intravaginal device at a rate of 0.1 mg/day to 20 mg/day, 0.5 mg/day to 15 mg/day, 1 mg/day to 10 mg/day, 2 mg/day to 8 mg/day, 4 mg/day to 6 mg/day, or 5 mg/day. In some embodiments, the anticholinergic agent is released from the intravaginal device at an average rate of 6 mg/day. In some embodiments, the anticholinergic agent is released from the intravaginal device at an average rate of 4 mg/day. In some embodiments, the anticholinergic agent is released, from the intravaginal device at an average rate of 2 mg/day.

In some embodiments, the first matrix of the intravaginal device of the present invention determines or controls the rate of release of an anticholinergic agent contained therein. In some embodiments, the second matrix of the intravaginal device determines or controls the rate of release of the anticholinergic agent. In some embodiments, both the first and second matrices determine or control the rate of release of the anticholinergic agent.

In some embodiments, the rate of release of the anticholinergic agent is dependent on the amount of halogenated siloxane polymer in the first matrix. In some embodiments, the release rate of the anticholinergic agent from the device is controlled by controlling the degree of cross-linking present in the polymer material of the first matrix. While not being bound to any particular theory, a high degree of cross-linking would be expected to result in a lower rate of release of the anticholinergic agent from the polymer matrix. The degree of crosslinking is controlled by the amount of crosslinker or catalyst used during production of the intravaginal device. See, e.g., U.S. Pat. No. 6,394,094.

In some embodiments, the release rate of the anticholinergic agent is controlled by the amount of siloxane polymer in the second matrix. In some embodiments, the release rate is controlled by both the amount of halogenated siloxane polymer in the first matrix and the amount siloxane polymer in the second matrix, wherein the siloxane polymer of the second matrix is a different polymer than the polymer of the first matrix.

In some embodiments, the release rate of the anticholinergic agent from the intravaginal device can also be controlled or modulated through the inclusion of additional agents or excipients in the polymer matrix, such as, for example, mineral oil, or fatty acid esters. In some embodiments, the release rate of the anticholinergic agent is controlled by the concentration of the anticholinergic agent in the second matrix.

In some embodiments, the release rate of the anticholinergic agent from the device is controlled by the volume of the pocket, the shape of the pocket, the thickness of the pocket wall, the degree by which the pocket wall encompasses the pocket, and/or the width of the slit in the first matrix.

In some embodiments, the invention is directed to a intravaginal device for decreasing the severity or the frequency of urinary urgency. In some embodiments, urinary urgency is characterized as the sudden, difficult to deter, and/or compelling desire to void urine.

In some embodiments, the device of the present invention allows for elimination of first-pass metabolism of the anti-cholinergic agent, e.g., oxybutynin, in the liver, thereby providing an advantage of the vaginal delivery of the present invention. Vaginal delivery can reduce the production of first-pass oxybutynin metabolite N-desethyloxybutynin, in some embodiments, reduction in the plasma concentration of this metabolite using the device of the present invention can reduce the severity of anticholinergic side effects, e.g., dry mouth, constipation, and/or blurred vision.

In some embodiments, the present invention provides a device for long-term delivery of a constant level of an anticholinergic agent, e.g., oxybutynin, from a single treatment.

In some embodiments, vaginal delivery device of the anticholinergic agent, e.g., oxybutynin, may allow accumulation of the anticholinergic agent at the bladder at lower doses than is achievable by oral dosing. While not being bound by any particular theory, the bladder and the vaginal tract are anatomically proximal to each other, and the vascular and lymphatic networks of the two organs are shared to a high degree, raising the possibility of accumulation of the anticholinergic agent at the bladder, During intravascular delivery, such accumulation in the bladder may enhance and/or prolong the therapeutic effects of the anticholinergic agent, allowing for decreased overall dosing of the anticholinergic agent.

The present invention is further illustrated by the following Examples. These Examples are provided to aid. In the understanding of the invention and are not to be construed as a limitation thereof.

### EXAMPLES

### Example 1

### Production of a First Matrix Vaginal Ring

A vaginal ring comprising a first matrix was prepared as follows. The first matrix was prepared using trifluoropropylmethyl/dimethyl siloxane. 40 g part A and 40 g part B trifluoropropylmethyl/dimethyl siloxane elastomer formation (NuSil Technology, CF2-3521 grade, Toms River, N.J.) were weighed into a 100 g capacity Hauschild mixing cup and subsequently mixed for 10 seconds in a Hauschild Model 501 T speed mixer. A metal spatula was then used to scrape down the sides of the mixing cup and further blend the two starting components. A final 14-second speed mixer cycle was supplied to ensure blend uniformity.

Two halves of an insert mold capable of forming a pocket and a pocket wall having a uniform thickness, were lightly coated in an ethanol/water solution of DARVAN WAQ (R.T. Vanderbilt Co., Norwalk, Conn.) and allowed to air dry. Between 12-15 grams of the 1:1 part A:part B blend were placed into the pin containing half of the mold. The insert pins were positioned in the filled portion of the mold and matched unfilled mold half was mated into place.

The filled mold assembly was then compressed between the unheated platens of a Kuntz injection molding machine in order to discharge excess polymer blend from the mold. During this compression step, the insert pins were held in place to avoid ejection by the applied air pressure. The discharged blend material was removed from the outside of the mold assembly and discarded.

The compressed, filled mold assembly was then placed between the preheated platens of a model 3912 Carver press. A pressure of 5,000 psi was applied and heating of the assembly for 15 minutes at 150° C. was performed to affect elastomer cure. During approximately the first 5 minutes of this curing step, the insert pins were held in place to avoid ejection from the mold.

After 15 minutes at 150° C., the mold was removed from the Carver press and cooled on the Kuntz machine's chiller for a sufficient time to allow easy separation of the mold halves and facilitate handling. The cured ring was separated from the mold. The insert pins were then carefully removed from the molded part by gently pulling them out without tearing or otherwise deforming the pocket.

This process resulted in a vaginal ring formed by mold compression haying an annular first matrix comprising a pocket and a pocket wall, wherein the pocket wall has a uniform thickness, and wherein the pocket wall encompasses the pocket.

### Example 2

### Production of a Two-Matrix Vaginal Ring

The pocket of the annular first matrix of a trifluoropropylmethyl/dimethyl siloxane elastomer prepared according to Example 1 was filled with a silicone/oxybutynin second matrix.

To form the second matrix, a mixture of 55% silicone and 45% oxybutynin was weighed in a Hauschild mixing cup and mixed in a Hauschild model AM 501 T speed mixer. A sufficient amount of the resulting silicone/oxybutynin paste was injected via syringe into the pocket of the ring of Example 1. In order to achieve a vaginal ring which released 4 mg/day oxybutynin, a vaginal ring comprising a first matrix having an outer diameter of 58.3 mm with a pocket that extended 80° around the exterior perimeter of the ring was used. The pocket had a diameter of 5.3 mm and was filled via syringe with the silicone/oxybutynin mixture. In order to achieve a vaginal ring which released 6 mg/day oxybutynin, a vaginal ring comprising a first matrix having an outer diameter of 58.3 mm with a pocket that extended 120° around the exterior perimeter of the ring was used. The pocket had a diameter of 5.3 mm. The ring was cured for 24 hours at ambient conditions to allow the silicone/oxybutynin polymer paste to solidify. The second matrix was held in the pocket of the first matrix by the pocket wall extending over the lateral surface area of the pocket. The silicone/oxybutynin mixture cured into a white cylindrically shaped solid, following the shape of either the 80° or 120° pocket.

This process resulted in an intravaginal ring having an annular first matrix comprising a pocket and a pocket wall, wherein the pocket wall has a uniform thickness, and wherein the pocket wall encompasses the pocket and a second matrix comprising an oxybutynin/silicone mixture contained in the pocket.

### Example 3

### Pharmacokinetics and Drug Metabolism in Animals

A study was conducted to determine the levels of oxybutynin and its active metabolite, N-desethyloxybutynin, present in plasma following oral and intravaginal administration of oxybutynin in dogs. Results from this study are presented in Table 1.

TABLE 1
Oxybutynin Vaginal Ring vs Oxybutynin Chloride
oral Tablet: Dose Comparison of Cₘₐₓ and Tₘₐₓ

| | | |
|---|---|---|
| Dosage Form | Dose | Cₘₐₓ (ng/mL) |

| | | | |
|---|---|---|---|
| Oxybutynin | 8 x 5 | mg/day | 25.6 |
| Chloride tablet | 2 x 5 | mg/day | 17.90 |
| Oxybutynin | 2.5 | mg/day | 13.95 |
| vaginal ring | 6.0 | mg/day | 18.75 |

A 14 day study was conducted, where 8 young adult females were randomly assigned to 4 groups of 2 dogs each. Two dogs received an oral 10 mg dose of oxybutynin chloride daily (2×5 mg/day tablets) for 14 consecutive days. The remaining 6 dogs received an intravaginal ring as described in Example 2, designed to continuously release oxybutynin at a dose of 0, 2.5 or 6 mg/day for 14 consecutive days.

Oxybutynin was detected in the plasma of dogs who were administered oxybutynin either orally or vaginally at all intervals tested. The average maximum (Cₘₐₓ) plasma levels of oxybutynin were slightly higher and were achieved sooner in dogs with the 6 mg/day vaginal rings (approximately 18.75 ng/mL at 1.5 hours (h) after dosing) than in dogs given oxybutynin orally (approximately 17.9 ng/mL at 3 h after dosing). The Cₘₐₓ values achieved for the 2.5 mg/day vaginal rings were slightly lower (approximately 13.95 ng/mL at 1.5 h after dosing).

Plasma levels of oxybutynin were sustained for up to 96 h after insertion of the vaginal ring (approximately 4.4 ng/mL and 11.6 ng/mL for dogs with 2.5 and 6.0 mg/day vaginal ring, respectively), but decreased rapidly when administered orally (to ≦2.75 ng/ml at 8 h or more after dosing). This data suggests that the area under the curve (“AUC”) values achieved with the 6 mg/day oxybutynin vaginal rings are slightly higher than those achieved after oral administration of 10 mg/day of oxybutynin chloride.

The amount of N-desethyloxybutynin detected in the plasma was consistently low (less than 1 ng/mL) for dogs given either concentration of oxybutynin vaginal rings. In contrast, the amount of N-desethyloxybutynin detected. In plasma of dogs given oxybutynin chloride orally was generally similar to the amount of oxybutynin that was measured.

These findings suggest that the 6 mg/day oxybutynin vaginal rings delivered similar, but more sustained amounts of oxybutynin to the plasma than oral administration of 10 mg/day oxybutynin chloride, while plasma levels of N-desethyloxybutynin were consistently lower in the vaginal ring relative to the oral administration.

### Example 4

### Pharmacokinetics and Drug Metabolism in Humans

Two studies were conducted to measure plasma oxybutynin and N-desethyloxybutynin concentrations over 7 days after insertion of oxybutynin vaginal rings releasing oxybutynin 2 mg/day, 4 mg/day, and 6 ing/day (as described in Example 2) in 8 healthy women, aged 45 to 62 years. Results of these studies are shown in Table 2 and Table 3, respectively.

TABLE 2
Pharmacokinetic Parameters for Oxybutynin:
2 mg/day Oxybutynin Vaginal Ring Treatment
Group: Pharmacokinetic Evaluable Patients

| Parameters | N | Mean | SD | Median | Min-Max |
|---|---|---|---|---|---|
| Observed | | | | | |
| Cₘₐₓ (ng/mL) | 8 | 4.10 | 1.13 | 3.87 | 2.67-6.42 |
| Tₘₐₓ (h) | 8 | 84.00 | 18.14 | 96.00 | 48.00-96.00 |
| Estimated | | | | | |
| Cₛₛ (ng/mL) | 8 | 3.562 | 1.017 | 3.58 | 2.03-5.02 |
| tₛₛ (h) | 8 | 46.64 | 18.29 | 46.37 | 26.97-86.02 |
| AUCₛₛ(24 h) | 8 | 85.48 | 24.41 | 86.04 | 48.71-120.49 |
| (h × ng/mL) | | | | | |
| rate | 5 | 0.06 | 0.02 | 0.06 | 0.04-0.08 |

Tₘₐₓ—time to maximum concentration; Cₛₛ—concentration at steady state; tₛₛ—time to reach steady state; AUCₛₛ—area under the cure at steady state.

TABLE 3
Pharmacokinetic Parameters for Oxybutynin:
4 mg/day Oxybutynin Vaginal Ring Treatment
Group: Pharmacokinetic Evaluable Patients

| Parameters | N | Mean | SD | Median | Min-Max |
|---|---|---|---|---|---|
| Observed | | | | | |
| Cmax (ng/mL) | 7 | 10.66 | 10.26 | 7.61 | 4.95-33.80 |
| Tₘₐₓ (h) | 7 | 75.43 | 25.66 | 72.00 | 24.00-96.00 |
| Estimated | | | | | |
| Cₛₛ (ng/mL) | 7 | 9.29 | 7.26 | 7.24 | 4.54-25.36 |
| tₛₛ (h) | 7 | 89.35 | 64.84 | 71.29 | 15.70-218.28 |
| AUCₛₛ (24 h) | 7 | 222.89 | 174.25 | 173.74 | 108.99-608.52 |
| (h × ng/mL) | | | | | |
| rate | 5 | 0.05 | 0.06 | 0.03 | 0.01-0.15 |

Tₘₐₓ—time to maximum concentration; Cₛₛ—concentration at steady state; tₛₛ—time to reach steady state; AUCₛₛ—area under the cure at steady state.

Blood samples were drawn at designated time points over a period of 96 h and on Day 7. Pharmacokinetics data used in the analysis include values obtained through the 96 h time point. As indicated in Tables 2 and 3, the mean Cₘₐₓ for oxybutynin was 4.1 ng/mL (median 3.9 ng/mL) in the 2 mg/day oxybutynin vaginal ring treatment group and 10.7 ng/mL, (median 7.6 ng/mL) in the 4 mg/day oxybutynin vaginal ring treatment group. All patients in both treatment groups experienced an initial peak in their plasma oxybutynin concentrations between 1.5 h and 6 h.

For N-desethyloxybutynin, pharmacokinetic analysis identical to that completed for oxybutynin was undertaken. Results for the 2 mg/day oxybutynin vaginal ring and 4 mg/day oxybutynin vaginal ring treatment groups are presented in Tables 4 and 5, respectively.

TABLE 4
Pharmacokinetic Parameters for N-desethyloxybutynin: 2 mg/day
Oxybutynin Vaginal Ring Treatment Group: Pharmacokinetic
Evaluable Patients

| Parameters | N | Mean | SD | Median | Min-Max |
|---|---|---|---|---|---|
| Observed | | | | | |
| Cₘₐₓ (ng/mL) | 8 | 6.60 | 2.47 | 6.78 | 2.10-10.05 |
| Tₘₐₓ (h) | 8 | 75.00 | 15.38 | 72.00 | 48.00-96.00 |
| Estimated | | | | | |
| Cₛₛ (ng/mL) | 8 | 6.26 | 2.33 | 6.88 | 1.68-8.47 |
| tₛₛ (h) | 8 | 66.60 | 49.21 | 50.49 | 18.11-173.32 |
| AUCₛₛ (24 h) | 8 | 150.21 | 55.88 | 165.05 | 40.35-203.16 |
| (h × ng/mL) | | | | | |
| rate | 7 | 0.05 | 0.040 | 0.04 | 0.01-0.13 |

Tₘₐₓ —time to maximum concentration;
Cₛₛ —concentration at steady state;
tₛₛ —time to reach steady state;
AUCₛₛ —area under the cure at steady state.

TABLE 5
Pharmacokinetic Parameters for N-desethyloxybutynin: 4 mg/day
Oxybutynin Vaginal Ring Treatment Group: Pharmacokinetic
Evaluable Patients

| Parameters | N | Mean | SD | Median | Min-Max |
|---|---|---|---|---|---|
| Observed | | | | | |
| Cₘₐₓ (ng/mL) | 7 | 7.82 | 3.43 | 6.73 | 4.67-14.49 |
| Tₘₐₓ (h) | 7 | 82.29 | 18.88 | 96.00 | 48.00-96.00 |
| Estimated | | | | | |
| Cₛₛ (ng/mL) | 7 | 7.48 | 3.48 | 6.45 | 3.72-14.33 |
| tₛₛ (h) | 7 | 63.39 | 32.44 | 57.71 | 31.08-128.79 |
| AUCₛₛ (24 h) (h × ng/mL) | 7 | 179.49 | 83.46 | 154.90 | 89.26-343.84 |
| rate | 7 | 0.04 | 0.02 | 0.04 | 0.02-0.07 |

Tₘₐₓ —time to maximum concentration;
Cₛₛ —concentration at steady state;
tₛₛ —time to reach steady state;
AUCₛₛ —area under the cure at steady state.

Table 6 and Table 7, respectively, summarize the results of the analysis of the mean Cₘₐₓ for oxybutynin was 8.9 ng/mL (median 8.9 ng/mL) in the 6 mg/day oxybutynin vaginal ring treatment group.

TABLE 6
Pharmacokinetic Parameters for Oxybutynin Vaginal Ring 6 mg/day:
Pharmacokinetic Evaluable Patients

| Parameters | N | Mean | SD | Median | Min-Max |
|---|---|---|---|---|---|
| Observed | | | | | |
| Cₘₐₓ (ng/mL) | 8 | 8.90 | 1.84 | 8.94 | 6.31-11.80 |
| Tₘₐₓ (h) | 8 | 66.00 | 24.84 | 72.00 | 24.00-96.00 |
| Estimated | | | | | |
| Cₛₛ (ng/mL) | 8 | 7.59 | 1.56 | 7.64 | 5.28-9.49 |
| tₛₛ (h) | 8 | 23.66 | 9.78 | 22.55 | 13.14-41.63 |
| t (½ Cₛₛ) (h) | 8 | 2.63 | 4.12 | 1.21 | 0.61-12.76 |
| ^{A}UCₛₛ (24 h) | 8 | 182.06 | 37.45 | 183.35 | 126.65-227.76 |
| (h × ng/mL) | | | | | |
| rate | 8 | 0.11 | 0.04 | 0.11 | 0.055-0.18 |

TABLE 7
Pharmacokinetic Parameters for N-desethyloxybutynin Oxybutynin
Vaginal Ring 6 mg/day: Pharmacokinetic Evaluable Patients

| Parameters | N | Mean | SD | Median | Min-Max |
|---|---|---|---|---|---|
| Observed | | | | | |
| Cₘₐₓ (ng/mL) | 8 | 16.23 | 4.7802 | 16.70 | 7.79-22.48 |
| Tₘₐₓ (h) | 8 | 82.50 | 21.6927 | 96.00 | 36.00-96.00 |
| Estimated | | | | | |
| Cₛₛ (ng/mL) | 8 | 15.21 | 5.03 | 15.21 | 6.70-21.90 |
| tₛₛ (h) | 8 | 56.24 | 31.30 | 44.36 | 25.38-115.54 |
| t (½ Cₛₛ₎ (h) | 8 | 13.51 | 8.45 | 9.27 | 5.59-25.89 |
| AUCₛₛ(24 h) (h × | 8 | 365.04 | 120.63 | 365.10 | 160.78-525.63 |
| ng/mL) | | | | | |
| rate | 8 | 0.05 | 0.02 | 0.05 | 0.02-0.09 |

In these studies, seven patients experienced an initial peak in their plasma oxybutynin concentrations between 1 and 5 h. Higher concentrations of oxybutynin were reached relative to concentrations of N-desethyloxybutynin for up to approximately 4 hours after vaginal ring insertion. After 6 h, concentrations of N-desethyloxybutynin were higher than oxybutynin concentrations in most cases, and concentrations of N-desethyloxybutynin continued to gradually rise until 72 h, while oxybutynin concentrations stabilized after 48 h.

The combined pharmacokinetics data suggest that 6 mg/day oxybutynin vaginal rings show a modest increase in plasma concentration of oxybutynin (measured by Cₘₐₓ and Cₛₛ)) over 4 mg/day oxybutynin vaginal rings. The 6 mg/day oxybutynin vaginal rings is further associated with an increase in the plasma concentration of N-desethyloxybutynin over that of the 4 mg/day oxybutynin vaginal rings.

### Example 5

### Plasma Oxybutynin Concentrations from Vaginal Administration

A preliminary clinical trial compared median plasma oxybutynin concentrations from 2 mg/day, 4 mg/day, and 6 mg/day oxybutynin vaginal ring treatment groups over a 4 week period. Results are summarized in Table 8.

TABLE 8
Comparative Pharmacokinetics for 2 mg/day, 4 mg/day, and 6 day/mg
Oxybutynin Vaginal Ring Treatment Groups

| | 2 mg/day | 4 mg/day | 6 mg/day |
|---|---|---|---|
| | oxybutynin | oxybutynin | oxybutynin |
| | vaginal ring | vaginal ring | vaginal ring |
| Treatment | | | |
| Period 1 | | | |
| Week 1 | 2.53 ng/mL | 4.67 ng/mL | 6.33 ng/mL |
| Week 3 | 2.96 ng/mL | 4.28 ng/mL | 7.02 ng/mL |
| Week 4 | 2.50 ng/mL | 4.29 ng/mL | 6.93 ng/mL |
| Treatment | | | |
| Period 2 | | | |
| Week 4 | 2.51 ng/mL | 4.26 ng/mL | 7.00 ng/mL |

(Median plasma concentration of oxybutynin)

### Example 6

### Comparison of Steady State Oxybutynin and Metabolite Plasma Levels of Vaginal Administration Versus Oral and Transdermal Administration

A comparison of the steady state oxybutynin and metabolite plasma levels to those reported for the marketed overactive bladder (OAB) products OXYTROL® 3.9 mg/day (transdermal patch. Watson Pharmaceutical, Inc., Morristown, N.J.) and DITROPAN XL® 15 mg/day (extended release oral tablet, Ortho-McNeil-Janssen Pharmaceutical, Inc., Titusville, N.J.) was conducted in order to estimate efficacy and safety parameters. Results are presented in Table 9.

TABLE 9
Comparative Pharmacokinetics for Oxybutynin Vaginal Ring,
Extended Release Oxybutynin Chloride Oral Tablets and
Transdermal Oxybutynin

| | | | Ratio |
|---|---|---|---|
| | | | N-Desethyloxy |
| | | N-Desethyloxy- | butynin/ |
| | Oxybutynin | butynin | Oxybutynin |
| | Mean Cₛₛ | Mean Cₛₛ | (area under |
| | (ng/mL) | (ng/mL) | the curve) |
| Vaginal ring 2 mg/day | 3.6 | 6.3 | 1.8 |
| Vaginal ring 4 mg/day | 9.3 | 7.5 | 0.8 |
| Vaginal ring 6 mg/day | 7.6 | 15.2 | 2.0 |
| DITROPAN XL ® | 3.0-3.5 | 13.2-14.2 | 4.1 |
| oxybutynin | | | |
| chloride oral tablets | | | |
| OXYTROL ® | 3.1-5.4 | 3.8-6.3 | 1.2 |
| oxybutynin | | | |
| 3.9 mg/day | | | |

Pharmacokinetic data from the oxybutynin vaginal rings was compared to pharmacokinetic data published for DITROPAN XL® extended release oral tablets and the transdermal OXYTROL® system. The oxybutynin vaginal ring produced plasma level of oxybutynin comparable to or slightly higher than those reported for DITROPAN XL® and OXYTROL® (depending on the specific oxybutynin release rate for the vaginal ring being evaluated). Plasma levels of N-desethyloxybutynin in vaginal ring-treated patients were generally lower than those reported for DITROPAN XL® extended release tablets but higher than those reported for OXYTROL®. For the 4 mg/day oxybutynin vaginal ring, the steady state oxybutynin level was similar to that reported for OXYTROL® and DITROPAN XL®. The metabolite N-desethyloxybutynin level of the 4 mg/day oxybutynin vaginal ring was similar to OXYTROL® but substantially lower than the N-desethyloxybutynin level reported for DITROPAN XL®. For the 6 mg/day oxybutynin vaginal ring, the steady state oxybutynin level was higher than that produced by either the OXYTROL® 3.9 mg/day patch or DITROPAN XL® 15 mg/day tablet. The metabolite N-desethyloxybutynin level was higher for the 6 mg/day oxybutynin vaginal ring than OXYTROL® but was still lower than the N-desethyloxybutynin level produced by DITROPAN XL®. These findings are reflected in the area under the curve ratios of N-desethyloxybutynin:oxybutynin, where oxybutynin vaginal ring ratios were similar to the ratios reported for the transdermal system but substantially lower than ratios for the extended release tablets.

### Example 7

### Study of the Safety and Efficacy of 4 mg/Day and 6 mg/Day Oxybutynin Vaginal Ring

A randomized, placebo-controlled clinical trial was conducted to study the safety and efficacy of an oxybutynin vaginal ring releasing either 4 mg/day, 6 mg/day (as described in Example 2) or placebo for the treatment of overactive bladder in women who had symptoms of predominant or pure urge incontinence, urinary urgency, or increased urinary frequency.

445 subjects entered the Treatment Period. The study included, four periods: a Screening Period of up to two weeks, a single-blind three-week Placebo Run-In Period, a 12-week double-blind Treatment Period, and a two week Follow-up Period. There was one screening visit followed by 8 other clinic visits: two visits during the Placebo Run-In (Visit 1 (Placebo Run-In Week 1), Visit 2 (Placebo Run-In Week 3)) and five visits during the Treatment Period (Visit 3 (Baseline), Visit 4 (Treatment Week 1), Visit 5 (Treatment Week 4), Visit 6 (Treatment Week 8) and Visit 7 (Treatment Week 12)). There was a follow-up visit two weeks after the last Treatment Period visit (Visit 8 (Follow-up)). Randomization occurred at Visit 1 (start of single-blind Placebo Run-In) to ensure that subjects received visually matching Placebo and Treatment period vaginal rings. The subjects were separated into three treatment groups, either the 4 mg/day oxybutynin vaginal ring group, the 6 mg/day oxybutynin vaginal ring group, or a placebo vaginal ring group.

During the study, four vaginal rings were inserted. Each used vaginal ring was replaced by a new vaginal ring at a scheduled time. Ring 1 was inserted at the start of Placebo Run-in period. Insertion was maintained throughout the three week Placebo Run-In period. Ring 2 was inserted at Visit 3 (Baseline). The vaginal ring was replaced one month thereafter: Ring 3 was inserted at Visit 5 (Treatment Week 4) and Ring 4 was inserted at Visit 6 (Treatment Week 8). This final vaginal ring was removed at Visit 7 (Treatment Week 12/Premature Discontinuation).

384 subjects (132 on the 4 mg/day oxybutynin vaginal ring, 119 on the 6 mg/day oxybutynin vaginal ring, and 133 on placebo vaginal ring) were included in the intention-to-treat (ITT) cohort, having provided baseline data and at least one valid post-baseline assessment of the number of incontinence episodes. The modified intent-to-treat cohort (MITT) consisted of ITT patients who met all three criteria for the definition of overactive bladder at baseline (Visit 3), i.e., predominant or pure urge incontinence consisting of ≧10 pure or predominant discrete urge incontinence episodes per week, and average urinary frequency of ≧8 voids per 24 hours and average total void of ≦3.9 L per 24 hours. The MITT cohort included 323 subjects. The PPC cohort further excluded patients with significant protocol deviations. Among the 384 ITT patients, 61 patients were excluded from the MITT cohort because they failed to meet at least one of the criteria at baseline.

Dose selection for this study was established by pharmacokinetic studies conducted with the oxybutynin vaginal ring at doses of 2 mg/day, 4 mg/day, and 6 mg/day. See Examples 4 and 5.

The primary measure of efficacy was the change from Visit 3 (Baseline) to Visit 7 (Treatment Week 12 Premature Discontinuation) in the total weekly number of incontinence episodes (stress plus urge), calculated by converting the total number of incontinence episodes (stress plus urge) occurring during the 3 consecutive OAB diary days prior to Visits 3 and 7 to a weekly-based number of episodes. Secondary efficacy measurements included the change from Visit 3 (Baseline) to Visit 7 (Treatment Week 12/Premature Discontinuation) for the following: average daily urinary frequency, the proportion of subjects with no incontinence episodes recorded in the final 3-day diary, the average void volume, and average severity of urgency.

The baseline characteristics number and percentage of subjects assigned to each of the analysis cohorts by treatment group are shown in Table 10.

TABLE 10
Subject Baseline Characteristics

| | Placebo | Oxy 4 mg | Oxy 6 mg | Total |
|---|---|---|---|---|
| Intent-to-Treat (ITT) | 133 | 132 | 119 | 384 |
| Modified ITT (MITT) | 112 | 115 | 96 | 323 |
| Exclusion from MITT* | 21 | 17 | 23 | 61 |
| Baseline Incontinence | 10 | 7 | 8 | 25 |
| Baseline Urinary Frequency | 5 | 8 | 8 | 21 |
| Baseline Void Volume | 7 | 2 | 8 | 17 |
| Per-Protocol Completers (PPC) | 71 | 81 | 64 | 216 |
| Exclusion from PPC* | 41 | 34 | 32 | 107 |
| Did Not Complete Study | 7 | 5 | 6 | 18 |
| Visit 7 Occurred Before | 3 | 1 | 0 | 4 |
| Day 74 | | | | |
| Use of Prohibited | 34 | 28 | 24 | 86 |
| Medications** | | | | |
| Protocol Deviations | 2 | 7 | 5 | 14 |

*A subject may be excluded due to more than one deviation.
**Based on verified list of prohibited medications.

Among the 384 ITT subjects, 61 subjects (15.9%) were excluded from the MITT cohort because they failed to meet at least one of the following criteria at baseline: >10 incontinence episodes per week, an average urinary frequency<8 voids per day, and an average total void volume<3.0 liters per day. A total of 25 of the 61 excluded subjects (41%) had <10 incontinence episodes at baseline, 21 subjects (34.4%) had urinary frequency of <8 voids per day, and 17 subjects (27.9%) had void volume>3.0 liters per day.

The Per-Protocol Completers (PPC) cohort consisted of 56.3% of the number of subjects included in the ITT cohort (216 PPC compared to 384 ITT subjects) and 66.9% of the number of MITT subjects (216 of 323 MITT subjects). Subjects excluded from the PPC Cohort (86 subjects) included those who violated study procedures.

Table 11 summarizes the results of the analysis of the mean reduction in the number of incontinence episodes from baseline to the end of treatment for the ITT cohort.

TABLE 11
Primary Outcome Analysis - ITT Cohort: Total Weekly Number of
Incontinence
Episodes: Change from Baseline (Visit 3) to End-of-Treatment (Visit 7)

| | | Base- | Mean | Standard | Differ- | P- |
|---|---|---|---|---|---|---|
| Treatments | N | line | Change* | Deviation | ence** | Value*** |
| OXY 4 mg | 132 | 26.34 | −15.38 | 16.12 | −2.22 | 0.0613 |
| OXY 6 mg | 119 | 25.12 | −15.18 | 16.24 | −2.02 | 0.1850 |
| Placebo | 133 | 26.44 | −13.16 | 14.65 | | |
| Treated | | | | | | |

*Change = Change in Total Weekly Number of Incontinence Episodes (Visit 3 to Visit 7 (or End-of-Treatment)).
**Difference = Difference between active treatment group and placebo.
***P-Value: Significance between active treatment groups and placebo was tested on raw data analysis.

Results show both the 4 mg/day oxybutynin vaginal ring and 6 mg/day oxybutynin vaginal ring groups had greater mean reductions in the total weekly number of incontinence episodes than the placebo vaginal ring group; for the 4 mg/day oxybutynin vaginal ring group, this result approached significance (p=0.0613). The treatment effect observed for the 6 mg/day oxybutynin vaginal ring was approximately the same as the 4 mg/day oxybutynin vaginal ring.

Any subject with qualifying values at baseline for all three principal inclusion criteria (>10 incontinence episodes per week, an average urinary frequency≧8 voids per day, and an average total void volume≦3.0 liters per day) could have been considered as presenting with an etiology of pure urgency. Therefore, in an additional evaluation of the number of incontinence episodes, defined prior to breaking the blind and before finalizing the study database, an MITT (Modified Intent-to-Treat) cohort, that included, this specific group of subjects, was defined. Although not considered the principal cohort for the evaluation of efficacy, the MITT cohort could be viewed as the most representative sample of subjects with OAB since it encompassed that group with the most well-defined set of attributes associated with a clinical presentation of OAB for clinical trials of new treatments.

Table 12 highlights the efficacy analysis of the reduction in the number of incontinence episodes from baseline to the end of treatment for the MITT cohort.

TABLE 12
Primary Outcome Analysis - Modified MITT Group Cohort:
Total Weekly Number of Incontinence Episodes: Change from Baseline
(Visit 3) to End-of-Treatment (Visit 7)

| | | Base- | Mean | Standard | Differ- | P- |
|---|---|---|---|---|---|---|
| Treatments | N | line | Change* | Deviation | ence** | Value*** |
| Oxy 4 mg | 115 | 28.34 | −16.76 | 16.45 | −2.99 | 0.0364 |
| Oxy 6 mg | 96 | 26.52 | −16.70 | 14.30 | −2.93 | 0.0176 |
| Placebo | 112 | 28.25 | −13.77 | 14.50 | | |

*Change = Change in Total Weekly Number of Incontinence Episodes (Visit 3 to Visit 7 (or End-of-Treatment)).
**Difference = Difference between active treatment group and placebo.
***P-Value: Significance between active treatment groups and placebo was tested on raw data analysis.

Results suggest statistically significant treatment effects favoring the 4 mg/day and 6 mg/day oxybutynin vaginal rings over placebo in this highly symptomatic group of subjects, with the 6 mg/day oxybutynin vaginal ring exhibiting an effect that is the same as that observed for the 4 mg/day oxybutynin vaginal ring group. Thus, the lower dose of 4 mg/day was sufficient to reduce the number of total weekly incontinence episodes. The MITT cohort results may represent the most clinically meaningful outcome associated with the oxybutynin vaginal ring because subjects in this cohort met the protocol-specified definition of clinical signs and symptoms of primarily urge incontinence, i.e., at baseline (Visit 3), all MITT subjects met the required criteria for the weekly number of incontinence episodes, urinary frequency, and void volume.

The PPC cohort summary statistics support the observed treatment effects for both active oxybutynin rings doses, with the observation that the 6 mg/day ring vaginal ring appears to provide no incremental benefit above that seen for the 4 mg/day vaginal ring.

Table 13 and 14 present descriptive statistics for the ITT cohort by menopausal status. The randomization was stratified by menopausal status, but subset analysis of each group was not planned. Therefore, although p-values were calculated, they were not based on any pre-specified hypothesis. The number of pre-menopausal patients in the study was substantially fewer than the number of menopausal patients.

For pre-menopausal patients, the patients in the 6 mg/day oxybutynin vaginal ring group and placebo group responded similarly, while patients in the 4 mg/day oxybutynin vaginal ring group did not see as great a decrease in total number of incontinence episodes.

TABLE 13
Primary Outcome Analysis (Pre-menopausal Patients) - ITT Cohort:
Total Weekly Number of Incontinence Episodes: Change from
Baseline (Visit 3) to End of Treatment (Visit 7)

| | | | Mean | Standard | |
|---|---|---|---|---|---|
| Treatments | N | Baseline | Change* | Deviation | Difference** |
| 4 mg/day | 35 | 30.53 | −14.73 | 19.49 | 2.38 |
| oxybutynin ring | | | | | |
| 6 mg/day | 25 | 28.00 | −17.55 | 18.00 | −0.44 |
| oxybutynin ring | | | | | |
| Placebo | 30 | 27.84 | −17.11 | 15.88 | |

*Change = Change in total weekly number of Incontinence Episodes (Visits 3 to Visit 7).
**Difference = Difference between active treatment group and placebo.

Menopausal patients demonstrated a larger reduction in total number of incontinence episodes when randomized to 4 mg/day and 6 mg/day oxybutynin vaginal rings as opposed to placebo.

TABLE 14
Primary Outcome Analysis (Menopausal Patients) - ITT Cohort:
Total Weekly Number of Incontinence Episodes: Change from
Baseline (Visit 3) to End of Treatment (Visit 7)

| | | | Mean | Standard | |
|---|---|---|---|---|---|
| Treatments | N | Baseline | Change* | Deviation | Difference** |
| 4 mg/day | 97 | 24.82 | −15.61 | 14.82 | −3.60 |
| oxybutynin | | | | | |
| ring | | | | | |
| 6 mg/day | 94 | 24.35 | −14.55 | 15.78 | −2.54 |
| oxybutynin | | | | | |
| ring | | | | | |
| Placebo | 103 | 26.03 | −12.01 | 14.14 | |

*Change = Change in total weekly number of Incontinence Episodes (Visits 3 to Visit 7).
**Difference = Difference between active treatment group and placebo.

For MITT and PPC cohorts, pre-menopausal patients did not show any additional reduction in total number of incontinence episodes for the 4 mg/ml and 6 mg/day groups compared to placebo. Menopausal patients in the MITT and PPC cohorts continued to show differences in the reduction of total number of incontinence episodes for the 4 mg/day and 6 mg/day groups compared to placebo. See Tables 15 and 16.

TABLE 15
Primary Outcome Analysis (Pre-menopausal Patients) - MITT Cohort:
Total Number of Incontinence Episodes: Change from Baseline
(Visit 3) to End of Treatment (Visit 7)

| | | | Mean | Standard | |
|---|---|---|---|---|---|
| Treatments | N | Baseline | Change* | Deviation | Difference** |
| 4 mg/day | 28 | 33.75 | −16.33 | 21.10 | 1.40 |
| oxybutynin ring | | | | | |
| 6 mg/day | 21 | 29.44 | −17.89 | 19.16 | −0.16 |
| oxybutynin ring | | | | | |
| Placebo | 25 | 29.96 | −17.73 | 16.68 | |

*Change = Change in total number of Incontinence Episodes (Visits 3 to Visit 7).
**Difference = Difference between active treatment group and placebo.

TABLE 16
Primary Outcome Analysis (Menopausal Patients) - MITT Cohort:
Total Number of Incontinence Episodes: Change from Baseline
(Visit 3) to End of Treatment (Visit 7)

| | | | Mean | Standard | |
|---|---|---|---|---|---|
| Treatments | N | Baseline | Change* | Deviation | Difference** |
| 4 mg/day | 87 | 26.61 | −16.90 | 14.79 | −4.27 |
| oxybutynin ring | | | | | |
| 6 mg/day | 75 | 25.70 | −16.36 | 12.64 | −3.73 |
| oxybutynin ring | | | | | |
| Placebo | 87 | 27.76 | −12.63 | 13.71 | |

*Change = Change in total number of Incontinence Episodes (Visits 3 to Visit 7).
**Difference = Difference between active treatment group and placebo.

Table 17 summarizes the findings associated with analysis for the total weekly number of incontinence episodes in the ITT cohort at each individual study visit. For the 4 mg/day oxybutynin vaginal ring, an observable treatment effect at day 28 (Visit 5) is slightly increasing at day 56 (Visit 6). This effect decreases somewhat at day 84 (Visit 7). A similar result was observed from MITT cohort. For 6 mg/day oxybutynin vaginal ring, the initial treatment effect at day 28 was somewhat smaller at day 56, but then increased substantially at the end of treatment, for both ITT and MITT cohorts.

TABLE 17
Secondary Outcome Analysis - ITT Cohort:
Total Weekly Number of Incontinence Episodes (stress plus urge):
Change from Baseline (Visit 3) to Subsequence Visit

| Change | | | | Stand- | | |
|---|---|---|---|---|---|---|
| from | | | | ard | | |
| Base- | | | Mean | Devi- | Differ- | P- |
| line to | Treatments | N | Change* | ation | ence** | value*** |
| Day 28/ | 4 mg/day | 119 | −12.33 | 13.964 | −2.43 | 0.2553 |
| Visit 5 | oxybutynin | | | | | |
| | ring | | | | | |
| | 6 mg/day | 101 | −13.01 | 13.406 | −3.11 | 0.0824 |
| | oxybutynin | | | | | |
| | ring | | | | | |
| | Placebo | 115 | −9.90 | 13.406 | | |
| Day 56/ | 4 mg/day | 118 | −14.83 | 14.816 | −2.67 | 0.0997 |
| Visit 6 | oxybutynin | | | | | |
| | ring | | | | | |
| | 6 mg/day | 107 | −13.26 | 15.208 | −1.10 | 0.1252 |
| | oxybutynin | | | | | |
| | ring | | | | | |
| | Placebo | 118 | −12.16 | 13.540 | | |

*Change = Change in total weekly number of Incontinence Episodes (Visits 3 to subsequent visits).
**Difference = Difference between active treatment group and placebo.
***P-value = Significance between active treatment group and placebo was tested on raw data analysts.

Table 18 and Table 19 summarize the findings of the total number of urge incontinence episodes for the ITT and MITT cohorts, respectively.

TABLE 18
Secondary Outcome Analysis - ITT Cohort:
Total Number of Urge Incontinence Episodes: Change from Baseline
(Visit 3) to End-of-Treatment (Visit 7)

| | | Base- | Mean | Standard | Differ- | P- |
|---|---|---|---|---|---|---|
| Treatments | N | line | Change* | Deviation | ence** | Value*** |
| OXY 4 mg | 132 | 24.18 | −15.13 | 15.393 | −2.80 | 0.0558 |
| OXY 6 mg | 119 | 23.06 | −14.90 | 14.950 | −2.57 | 0.1803 |
| Placebo | 133 | 23.88 | −12.43 | 14.311 | | |
| Treated | | | | | | |

*Change = Change in Total Number of Urge Incontinence Episodes (Visit 3 to Visit 7 (or End-of-Treatment)).
**Difference = Difference between active treatment group and placebo.
***P-Value: Significance between active treatment groups and placebo was tested on raw data analysis.

TABLE 19
Secondary Outcome Analysis - MITT Cohort:
Total Number of Urge Incontinence Episodes: Change from Baseline
(Visit 3) to End-of-Treatment (Visit 7)

| | | Base- | Mean | Standard | Differ- | P- |
|---|---|---|---|---|---|---|
| Treatments | N | line | Change* | Deviation | ence** | Value*** |
| Oxy 4 mg | 115 | 25.99 | −16.37 | 15.753 | −3.29 | 0.0544 |
| Oxy 6 mg | 96 | 24.28 | −16.38 | 13.380 | −3.30 | 0.0223 |
| Placebo | 112 | 25.63 | −13.08 | 14.439 | | |

*Change = Change in Total Number of Urge Incontinence Episodes (Visit 3 to Visit 7 (or End-of-Treatment)).
**Difference = Difference between active treatment group andplacebo.
***P-Value: Significance between active treatment groups and placebo was tested on raw data analysis.

Both treatment groups demonstrated a reduction in the weekly number of urge-only incontinence episodes to a greater extent than the placebo group. Compared to placebo, the 4 mg/day oxybutynin vaginal ring (p=0.0558 for the ITT and p=0.0544 for the MITT cohort) experienced fewer urge-only incontinence episodes while the 6 mg/day oxybutynin vaginal ring in the MITT cohort (p=0.0223) experienced fewer urge-only incontinence episodes. As indicated for the total incontinence episode endpoint, the 6 mg/day oxybutynin vaginal ring provided no additive treatment effect compared to the 4 mg/day oxybutynin vaginal ring, but both oxybutynin vaginal rings demonstrated a greater magnitude of reduction of urge-only episodes compared to placebo for the MITT cohort (a differential reduction of 3.3 episodes greater than what was observed for placebo).

The analysis of urge incontinence episodes was investigated by menopausal status and is presented in Tables 20 and 21 for the MITT cohort. Results were consistent with what was observed when considering the primary efficacy endpoint, the total weekly number of incontinence episodes. The magnitude of the difference in the mean reduction of urge-only incontinence episodes was greater for both oxybutynin vaginal rings groups in the MITT cohort compared to the ITT cohort.

TABLE 20
Secondary Outcome Analysis (Pre-Menopausal Patients) -
MITT Cohort: Total Number of Urge Incontinence Episodes:
Change from Baseline (Visit 3) to End-of-Treatment (Visit 7)

| | | | Mean | Standard | |
|---|---|---|---|---|---|
| Treatments | N | Baseline | Change* | Deviation | Difference** |
| Oxy 4 mg | 28 | 30.17 | −15.83 | 19.864 | −0.06 |
| Oxy 6 mg | 21 | 27.33 | −17.44 | 18.893 | −1.67 |
| Placebo | 25 | 27.16 | −15.77 | 16.924 | |

*Change = Change in Total Number of Urge Incontinence Episodes (Visit 3 to Visit 7 (or End-of-Treatment)).
**Difference = Difference between active treatment group and placebo.

TABLE 21
Secondary Outcome Analysis (Menopausal Patients) -
MITT Cohort: Total Number of Urge Incontinence Episodes:
Change from Baseline (Visit 3) to End-of-Treatment (Visit 7)

| | | | Mean | Standard | |
|---|---|---|---|---|---|
| Treatments | N | Baseline | Change* | Deviation | Difference** |
| Oxy 4 mg | 87 | 24.65 | −16.55 | 14.316 | −4.24 |
| Oxy 6 mg | 75 | 23.43 | −16.08 | 11.531 | −3.77 |
| Placebo | 87 | 25.18 | −12.31 | 13.655 | |

*Change = Change in Total Number of Urge Incontinence Episodes (Visit 3 to Visit 7 (or End-of-Treatment)).
**Difference = Difference between active treatment group and placebo.

Tables 22 and 23 summarize the findings associated with analysis for the total weekly number of urge incontinence episodes in the ITT and MITT cohorts, respectively, at individual study visits. In both cohort analyses, 4 mg/day oxybutynin vaginal rings were shown to provide a relatively consistent reduction in the weekly number of urge-only episodes compared to placebo that continued through to the end of treatment. For 6 mg/day oxybutynin vaginal ring, an initial larger differential effect was observed at day 28 then diminished at day 56, which then rebounded somewhat at the end of treatment. The 6 mg/day reduction overall, however, was no greater than that observed for the 4 mg/day group.

TABLE 22
Secondary Outcome Analysis - ITT Cohort:
Total Weekly Number of Incontinence Episodes (urge only):
Change from Baseline (Visit 3) to subsequence visits

| Change | | | | | | |
|---|---|---|---|---|---|---|
| from | | | Mean | Standard | | P- |
| Baseline to | Treatments | N | Change* | Deviation | Difference** | value*** |
| Day 28/ | 4 mg/day | 119 | −11.90 | 14.178 | −2.87 | 0.2926 |
| Visit 5 | oxybutynin ring | | | | | |
| | 6 mg/day | 101 | −13.65 | 12.947 | −4.62 | 0.0286 |
| | oxybutynin ring | | | | | |
| | Placebo | 115 | −9.03 | 13.277 | | |
| Day 56/ | 4 mg/day | 118 | −14.47 | 14.005 | −3.08 | 0.0501 |
| Visit 6 | oxybutynin ring | | | | | |
| | 6 mg/day | 107 | −13.69 | 13.273 | −2.30 | 0.0221 |
| | oxybutynin ring | | | | | |
| | Placebo | 118 | −11.39 | 13.080 | | |

*Change = Change in total weekly number of Incontinence Episodes (urge only) (Visits 3 to subsequent visits).
**Difference = Difference between active treatment group and placebo.
***P-value = Significance between active treatment group and placebo was tested on raw data analysts.

TABLE 23
Secondary Outcome Analysis - MITT Cohort:
Total Weekly Number of Incontinence Episodes (urge only):
Change from Baseline (Visit 3) to subsequence visits

| Change | | | | | | |
|---|---|---|---|---|---|---|
| from | | | Mean | Standard | | P- |
| Baseline to | Treatments | N | Change* | Deviation | Difference** | value*** |
| Day 28/ | 4 mg/day | 103 | −12.57 | 14.873 | −3.09 | 0.0669 |
| Visit 5 | oxybutynin ring | | | | | |
| | 6 mg/day | 83 | −14.56 | 12.804 | −5.08 | 0.0042 |
| | oxybutynin ring | | | | | |
| | Placebo | 98 | −9.48 | 13.530 | | |
| Day 56/ | 4 mg/day | 103 | −15.50 | 14.346 | −3.28 | 0.0359 |
| Visit 6 | oxybutynin ring | | | | | |
| | 6 mg/day | 87 | −14.43 | 13.091 | −2.21 | 0.0144 |
| | oxybutynin ring | | | | | |
| | Placebo | 101 | −12.22 | 12.734 | | |

*Change = Change in total weekly number of Incontinence Episodes (urge only) (Visits 3 to subsequent visits).
**Difference = Difference between active treatment group and placebo.
***P-value = Significance between active treatment group and placebo was tested on raw data analysts.

Table 24 summarizes the findings associated with the analysis of the change from baseline to end-of-treatment for the average daily urinary frequency in the subjects who were treated.

TABLE 24
Secondary Outcome Analysis - ITT Cohort: Average Daily Urinary
Frequency: Change from Baseline (Visit 3) to End-of-Treatment (Visit 7)

| Treat- | | Base- | Mean | Standard | Differ- | P- |
|---|---|---|---|---|---|---|
| ments | N | line | Change* | Deviation | ence** | Value*** |
| OXY 4 mg | 132 | 11.36 | −1.70 | 2.806 | −0.60 | 0.0722 |
| OXY 6 mg | 119 | 10.82 | −2.03 | 2.771 | −0.93 | 0.0004 |
| Placebo | 133 | 11.24 | −1.10 | 2.730 | | |
| Treated | | | | | | |

*Change = Change in Average Daily Urinary Frequency (Visit 3 to Visit 7 (or End-of-Treatment)).
**Difference = Difference between active treatment group and placebo.
***P-Value: Significance between active treatment groups and placebo was tested on raw data analysis.

All treatment groups demonstrated a statistically significant reduction in the average daily urinary frequency. In the ITT cohort, the 6 mg/day oxybutynin vaginal ring demonstrated a statistically significant reduction (p=0.0004) in average daily urinary frequency from baseline to end-of-treatment compared to placebo. The 4 mg/day oxybutynin vaginal ring also demonstrated reduction in average daily urinary frequency when compared to placebo that approached significance (p=0.0722).

Analysis for the MITT cohort (Table 25) yielded similar results.

TABLE 25
Secondary Outcome Analysis - MITT Cohort:
Average, Daily Urinary Frequency: Change from Baseline (Visit 3) to
End-of-Treatment (Visit 7)

| Treat- | | Base- | Mean | Standard | Differ- | |
|---|---|---|---|---|---|---|
| ments | N | line | Change* | Deviation | ence** | P-Value*** |
| Oxy 4 mg | 115 | 11.60 | −1.80 | 2.839 | −0.7 | 0.1039 |
| Oxy 6 mg | 96 | 11.01 | −2.10 | 2.918 | −1.0 | 0.0020 |
| Placebo | 112 | 11.32 | −1.10 | 2.746 | | |

*Change = Change in Average Daily Urinary Frequency (Visit 3 to Visit 7 (or End-of-Treatment)).
**Difference = Difference between active treatment group and placebo.
***P-Value: Significance between active treatment groups and placebo was tested on raw data analysis.

Analysis of average void volume in ml for the ITT and MITT cohorts is presented in Tables 26 and 27, respectively. In both cohorts, all three treatment groups showed very little difference in daily average void volume from baseline (Visit 3) to End-of-treatment. Neither the 4 mg/day nor the 6 mg/day significantly increased daily average void volume compared to placebo.

TABLE 26
Secondary Outcome Analysis - ITT Cohort:
Daily Average Void Volume: Change from Baseline (Visit 3) to End-of-
Treatment (Visit 7)

| Treat- | | Base- | Mean | Standard | Differ- | P- |
|---|---|---|---|---|---|---|
| ments | N | line | Change* | Deviation | ence** | Value*** |
| Oxy 4 mg | 131 | 1597.89 | −73.55 | 523.862 | 19.77 | 0.6300 |
| Oxy 6 mg | 117 | 1712.96 | −108.03 | 632.052 | −14.71 | 0.7372 |
| Placebo | 132 | 1750.64 | −93.32 | 646.620 | | |

*Change = Change in Average Daily Average Void Volume (Visit 3 to Visit 7 (or End-of-Treatment)).
**Difference = Difference between active treatment group and placebo.
***P-Value: Significance between active treatment groups and placebo was tested on raw data analysis.

TABLE 27
Secondary Outcome Analysis - MITT Cohort:
Daily Average Void Volume: Change from Baseline (Visit 3) to End-of-
Treatment (Visit 7)

| Treat- | | Base- | Mean | Standard | Differ- | P- |
|---|---|---|---|---|---|---|
| ments | N | line | Change* | Deviation | ence** | Value*** |
| Oxy 4 mg | 114 | 1630.60 | −100.75 | 487.074 | −58.86 | 0.3969 |
| Oxy 6 mg | 94 | 1632.90 | −55.42 | 587.371 | −13.53 | 0.8301 |
| Placebo | 111 | 1627.5 | −41.89 | 564.552 | | |

*Change = Change in Average Daily Void Volume (Visit 3 to Visit 7 (or End-of-Treatment)).
**Difference = Difference between active treatment group and placebo.
***P-Value: Significance between active treatment groups and placebo was tested on raw data analysis.

Table 28 summarizes the findings associated with analysis of the change from baseline to end-of-treatment for the average void volume per void in the subjects who were treated.

TABLE 28
Secondary Outcome Analysis - ITT Cohort:
Average Void Volume Per Void: Change from Baseline (Visit 3)
to End-of-Treatment (Visit 7)

| Treat- | | Base- | Mean | Standard | Differ- | P- |
|---|---|---|---|---|---|---|
| ments | N | line | Change* | Deviation | ence** | Value*** |
| OXY 4 mg | 131 | 53.06 | 5.19 | 15.398 | 3.44 | 0.2134 |
| OXY 6 mg | 117 | 59.49 | 7.07 | 19.821 | 5.32 | 0.0126 |
| Placebo | 132 | 58.63 | 1.75 | 16.981 | | |
| Treated | | | | | | |

*Change = Change in Average Void Volume Per Void (Visit 3 to Visit 7 (or End-of-Treatment)).
**Difference = Difference between active treatment group and placebo.
***P-Value: Significance between active treatment groups and placebo was tested on raw data analysis.

The 6 mg/day oxybutynin vaginal ring demonstrated a significantly greater increase in the average volume per void as compared to placebo. The 4 mg/day oxybutynin vaginal ring also demonstrated a reduction, although not significant, in the average volume per void as compared to placebo.

Tables 29 and 30 summarize the findings associated with analysis of the change from baseline to end-of-treatment for the average severity of urgency in the ITT and MITT cohorts, respectively.

TABLE 29
Secondary Outcome Analysis - ITT Cohort:
Average Severity of Urgency: Change from Baseline (Visit 3) to End-of-
Treatment (Visit 7)

| Treat- | | Base- | Mean | Standard | Differ- | P- |
|---|---|---|---|---|---|---|
| ments | N | line | Change* | Deviation | ence** | Value*** |
| OXY 4 mg | 132 | 18.78 | −3.59 | 6.648 | −1.01 | 0.2234 |
| OXY 6 mg | 118 | 17.90 | −4.38 | 6.493 | −1.80 | 0.0065 |
| Placebo | 133 | 18.57 | −2.58 | 5.663 | | |
| Treated | | | | | | |

*Change = Change in Average Daily Severity of Urgency (Visit 3 to Visit 7 (or End-of-Treatment)).
**Difference = Difference between active treatment group and placebo.
***P-Value: Significance between active treatment groups and placebo was tested on raw data analysis.

TABLE 30
Secondary Outcome Analysis - MITT Cohort:
Average Severity of Urgency: Change from Baseline (Visit 3) to End-of-
Treatment (Visit 7)

| Treat- | | Base- | Mean | Standard | Differ- | |
|---|---|---|---|---|---|---|
| ments | N | line | Change* | Deviation | ence** | P-Value*** |
| Oxy 4 mg | 115 | 19.26 | −3.59 | 6.505 | −1.16 | 0.2730 |
| Oxy 6 mg | 96 | 18.07 | −4.20 | 6.805 | −1.77 | 0.0261 |
| Placebo | 112 | 18.59 | −2.43 | 5.461 | | |

*Change = Change in Average Daily Severity of Urgency (Visit 3 to Visit 7 (or End-of-Treatment)).
**Difference = Difference between active treatment group and placebo.
***P-Value: Significance between active treatment groups and placebo was tested on raw data analysis.

In the ITT cohort, both oxybutynin vaginal ring groups showed differentially greater reductions compared to placebo; for 6 mg/day oxybutynin vaginal ring, this difference was statistically significant (p=0.0065). The MITT cohort gave similar results to the ITT cohort.

Tables 31 and 32 summarize the findings associated with analysis of the proportion of subjects with no incontinence episodes recorded in the Final 3-day diary at the end-of treatment visit for the ITT and MITT cohorts, respectively.

TABLE 31
Secondary Outcome Analysis - ITT Cohort: Proportion of
Subjects with no Incontinence Episodes Recorded in
Final 3-Day Diary

| Treatments | % | P-Value* |
|---|---|---|
| Oxy 4 mg | (35/132) 26.52% | 0.1476 |
| Oxy 6 mg | (35/119) 29.41% | 0.0602 |
| Placebo | (25/133) 18.80% | |

*Based on stratified Cochran-Mantel-Haenszel tests between active treatment and placebo.

TABLE 32
Secondary Outcome Analysis - MITT Cohort: Proportion of
Subjects with no Incontinence Episodes Recorded Fina
3-Day Diary

| Treatments | % | P-Value* |
|---|---|---|
| Oxy 4 mg | (29/115) 5.22% | 0.0258 |
| Oxy 6 mg | (25/96) 26.04% | 0.0269 |
| Placebo | (15/112) 13.39% | |

*Based on stratified Cochran-Mantel-Haenszel tests between active treatment and placebo.

In the ITT cohort, both 4 mg/day oxybutynin vaginal ring (26.52%) and 6 mg/day oxybutynin vaginal ring (29.41%) had larger proportions of subjects compared to placebo (18.80%) who reported no incontinence episodes at the end-of-treatment Visit. For the MITT cohort, the proportions of subjects reporting no incontinence episodes at the end of treatment was substantially less for subjects receiving placebo (13.39%), leading to statistically significant differences favoring both 4 mg/day oxybutynin vaginal ring (p=0.0258) and 6 mg/day oxybutynin vaginal ring (p=0.0269).

Visual Analogue Scale (VAS) was recorded using a 100 mm scale, marked off in 10 segments. One end of the scale had the anchor “absence of symptoms” while the other end had the anchor “unbearable symptoms.” The patients were asked to circle a line on the scale indicating the best reflection of her subjective symptoms associated with overactive bladder overlooking the time window of the last 4 weeks, with 1 being the best and 10 being the worst.

Results of the analysis in VAS from baseline (visit 3) to End-of-Treatment for the ITT cohort are present in Table 33. For the ITT cohort, both the 4 mg/day oxybutynin ring (p=0.0199) and the 6 mg/day oxybutynin ring (p=0.0012) achieved significance in reducing the VAS compared to placebo. Results were similar for the MITT cohort where both the 4 mg/day oxybutynin ring (p=0.0374) and the 6 mg/day oxybutynin rings (p=0.0045) achieved significance compared to placebo as well.

TABLE 33
Secondary Outcome Analysis - ITT Cohort:
VAS: Change from Baseline (Visit 3) to End-of-Treatment (Visit 7)

| Treat- | | Base- | Mean | Standard | Differ- | |
|---|---|---|---|---|---|---|
| ments | N | line | Change* | Deviation | ence** | P-Value*** |
| Oxy 4 mg | 131 | 5.77 | −1.79 | 2.903 | −0.52 | 0.0199 |
| Oxy 6 mg | 117 | 6.43 | −2.50 | 2.674 | −1.23 | 0.0012 |
| Placebo | 131 | 6.03 | −1.27 | 2.605 | | |

*Change = Change in VAS (Visit 3 to Visit 7 (or End-of-Treatment)).
**Difference = Difference between active treatment group and placebo.
***P-Value: Significance between active treatment groups and placebo was tested on raw data analysis.

Urinary Distress Inventory (UDI) was a list of 19 symptoms described by people who have bladder problems and/or who experience urine leakage. Patients filled out the UDI, indicating which symptoms they had experienced in the past 4 weeks and, of those, how bothersome they were. The scale to assess how bothersome the symptoms were ranged from 0 to 3, 0 for “not at all,” 1 for “slightly”, 2 for “moderately”, and 3 for “greatly.” Analysis results of the change from baseline (Visit 3) to end-of-treatment (Visit 7) for all 19 questions for the ITT cohort are presented below.

For the ITT cohort, statistically significant differences between the treatment groups and placebo were found in the assessment of the 6 different symptoms from the mean change from baseline (Visit 3) to end-of-treatment (Visit 7). Both the 4 mg/day and 6 mg/day oxybutynin vaginal rings achieved statistical significance compared to placebo for reducing the experience of frequent urination (4 mg/day p=0.0016, 6 mg/day p=0.0007), the strong feeling of urgency to empty bladder (4 mg/day p=0.0277, 6 mg/day p=0.0028), the experience of urine leakage related to the feeling of urgency (94 mg/day p=0.0091, 6 mg/day p 0.0025), the experience of small amounts of urine leakage (4 mg/day p=0.0056, 6 mg/day p=0.226), and the experience of large amounts of urinary leakage (4 mg/day p=0.0260, 6 mg/day p=0.0030). For the experience of nighttime urination, 4 mg/day (p=0.0100) achieved a significant reduction compared to placebo, whereas 6 mg/day (p=0.0732) approached significance. Tables 34-52 show the analysis of each question in the UDI for the ITT cohort.

TABLE 34
Secondary Outcome Analysis - ITT Cohort:
UDI - Did you Experience Frequent Urination?
Change from Baseline (Visit 3) to End-of-Treatment (Visit 7)

| Treat- | | Base- | Mean | Standard | Differ- | |
|---|---|---|---|---|---|---|
| ments | N | line | Change* | Deviation | ence** | P-Value*** |
| Oxy 4 mg | 130 | 1.88 | −0.76 | 1.112 | −0.31 | 0.0016 |
| Oxy 6 mg | 119 | 2.14 | −0.94 | 1.122 | −0.49 | 0.0007 |
| Placebo | 130 | 2.00 | −0.45 | 0.943 | | |

*Change = Change in severity of UDI - Did you Experience Frequent Urination? (Visit 3 to Visit 7 (or End-of-Treatment)).
**Difference = Difference between active treatment group and placebo.
***P-Value: Significance between active treatment groups and placebo was tested on raw data analysis.

TABLE 35
Secondary Outcome Analysis - ITT Cohort:
UDI - A Strong Feeling of Urgency to Empty Bladder?
Change from Baseline (Visit 3) to End-of-Treatment (Visit 7)

| Treat- | | Base- | Mean | Standard | Differ- | |
|---|---|---|---|---|---|---|
| ments | N | line | Change* | Deviation | ence** | P-Value*** |
| Oxy 4 mg | 131 | 1.79 | −0.55 | 1.097 | −0.2 | 0.0277 |
| Oxy 6 mg | 119 | 2.03 | −0.77 | 1.093 | −0.42 | 0.0028 |
| Placebo | 132 | 1.89 | −0.35 | 0.894 | | |

*Change = Change in severity of UDI - A Strong Feeling of Urgency to Empty Bladder? (Visit 3 to Visit 7 (or End-of-Treatment)).
**Difference = Difference between active treatment group and placebo.
***P-Value: Significance between active treatment groups and placebo was tested on raw data analysis.

TABLE 36
Secondary Outcome Analysis - ITT Cohort:
UDI - Did You Experience Urine Leakage Related to the Feeling of
Urgency?
Change from Baseline (Visit 3) to End-of-Treatment (Visit 7)

| Treat- | | Base- | Mean | Standard | Differ- | |
|---|---|---|---|---|---|---|
| ments | N | line | Change* | Deviation | ence** | P-Value*** |
| Oxy 4 mg | 132 | 1.76 | −0.73 | 1.173 | −0.25 | 0.0091 |
| Oxy 6 mg | 119 | 1.91 | −0.87 | 1.062 | −0.39 | 0.0025 |
| Placebo | 133 | 1.85 | −0.48 | 1.052 | | |

*Change = Change in severity of UDI - Did You Experience Urine Leakage Related to the Feeling of Urgency? (Visit 3 to Visit 7 (or End-of-Treatment)).
**Difference = Difference between active treatment group and placebo.
***P-Value: Significance between active treatment groups and placebo was tested on raw data analysis.

TABLE 37
Secondary Outcome Analysis - ITT Cohort:
UDI - Did You Experience Urine Leakage Related to Physical Activity,
Coughing or Sneezing?
Change from Baseline (Visit 3) to End-of-Treatment (Visit 7)

| Treat- | | Base- | Mean | Standard | Differ- | |
|---|---|---|---|---|---|---|
| ments | N | line | Change* | Deviation | ence** | P-Value*** |
| Oxy 4 mg | 132 | 0.76 | −0.14 | 0.898 | 0.1 | 0.5409 |
| Oxy 6 mg | 118 | 0.90 | −0.22 | 0.878 | 0.02 | 0.6632 |
| Placebo | 133 | 0.86 | −0.24 | 0.909 | | |

*Change = Change in severity of UDI - Did You Experience Urine Leakage Related to Physical Activity, Coughing or Sneezing? (Visit 3 to Visit 7 (or End-of-Treatment)).
**Difference = Difference between active treatment group and placebo.
***P-Value: Significance between active treatment groups and placebo was tested on raw data analysis.

TABLE 38
Secondary Outcome Analysis - ITT Cohort:
UDI - Did You Experience Urine Leakage Not Related to Urgency or
Activity? Change from Baseline (Visit 3) to End-of-Treatment (Visit 7)

| Treat- | | Base- | Mean | Standard | Differ- | |
|---|---|---|---|---|---|---|
| ments | N | line | Change* | Deviation | ence** | P-Value*** |
| Oxy 4 mg | 130 | 0.68 | −0.16 | 0.922 | −0.01 | 0.8660 |
| Oxy 6 mg | 119 | 0.71 | −0.34 | 1.020 | −0.19 | 0.1151 |
| Placebo | 131 | 0.65 | −0.15 | 0.949 | | |

*Change = Change in severity of UDI - Did You Experience Urine Leakage Not Related to Urgency or Activity? (Visit 3 to Visit 7 (or End-of-Treatment)).
**Difference = Difference between active treatment group and placebo.
***P-Value: Significance between active treatment groups and placebo was tested on raw data analysis.

TABLE 39
Secondary Outcome Analysis - ITT Cohort:
UDI - Did You Experience Small Amounts of Leakage (i.e., Drops)?
Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 130 | 1.42 | −0.57 | 1.092 | −0.34 | 0.0056 |
| Oxy 6 mg | 118 | 1.46 | −0.56 | 0.966 | −0.33 | 0.0226 |
| Placebo | 133 | 1.32 | −0.23 | 1.016 | | |

*Change = Change in severity of UDI - Did You Experience Small Amounts of Leakage (i.e., Drops)? (Visit 3 to Visit 7 (or End-of-Treatment)).
**Difference = Difference between active treatment group and placebo.
***P-Value: Significance between active treatment groups and placebo was tested on raw data analysis.

TABLE 40
Secondary Outcome Analysis - ITT Cohort:
UDI - Did You Experience Large Amounts of Urinary Leakage?
Change front Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 132 | 1.02 | −0.46 | 1.322 | −0.06 | 0.0200 |
| Oxy 6 mg | 119 | 1.25 | −0.70 | 1.239 | −0.3 | 0.0030 |
| Placebo | 132 | 1.32 | −0.40 | 1.043 | | |

*Change = Change in severity of UDI - Did You Experience Large Amounts of Urinary Leakage? (Visit 3 to Visit 7 (or End-of-Treatment)).
**Difference = Difference between active treatment group and placebo.
***P-Value: Significance between active treatment groups and placebo was tested on raw data analysis.

TABLE 41
Secondary Outcome Analysis - ITT Cohort:
UDI - Did You Experience Nighttime Urination
Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 130 | 1.54 | −0.55 | 1.057 | −0.25 | 0.0100 |
| Oxy 6 mg | 118 | 1.69 | −0.54 | 1.107 | −0.24 | 0.0732 |
| Placebo | 133 | 1.63 | −0.30 | 0.847 | | |

*Change = Change in severity of UDI - Did You Experience Nighttime Urination (Visit 3 to Visit 7 or End-of-Treatment)).
**Difference = Difference between active treatment group and placebo.
***P-Value: Significance between active treatment groups and placebo was tested on raw data analysis.

TABLE 42
Secondary Outcome Analysis - ITT Cohort:
UDI - Did You Experience Bed Wetting?
Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 132 | 0.17 | −0.05 | 0.537 | 0.03 | 1.0000 |
| Oxy 6 mg | 119 | 0.18 | −0.08 | 0.555 | 0 | 0.5169 |
| Placebo | 133 | 0.23 | −0.08 | 0.488 | | |

*Change = Change in severity of UDI - Did You Experience Bed Wetting? (Visit 3 to Visit 7 (or End-of-Treatment)).
**Difference = Difference between active treatment group and placebo.
***P-Value: Significance between active treatment groups and placebo was tested on raw data analysis.

TABLE 43
Secondary Outcome Analysis - ITT Cohort:
UDI - Did You Experience Difficulty Emptying Your Bladder?
Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 131 | 0.29 | −0.07 | 0.647 | −0.03 | 0.5941 |
| Oxy 6 mg | 119 | 0.24 | −0.03 | 0.559 | −0.01 | 0.7669 |
| Placebo | 133 | 0.29 | −0.04 | 0.558 | | |

*Change = Change in severity of UDI - Did You Experience Difficulty Emptying Your Bladder? (Visit 3 to Visit 7 (or End-of-Treatment)).
**Difference = Difference between active treatment group and placebo.
***P-Value: Significance between active treatment groups and placebo was tested on raw data analysis.

TABLE 44
Secondary Outcome Analysis - ITT Cohort:
UDI - Did You Experience Feeling of Incomplete Bladder Emptying?
Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 132 | 0.70 | −0.27 | 0.839 | −0.1 | 0.9700 |
| Oxy 6 mg | 119 | 0.66 | −0.32 | 0.712 | −0.15 | 0.2430 |
| Placebo | 133 | 0.55 | −0.17 | 0.746 | | |

*Change = Change in severity of UDI - Did You Experience Feeling of Incomplete Bladder Emptying? (Visit 3 to Visit 7 (or End-of-Treatment)).
**Difference = Difference between active treatment group and placebo.
***P-Value: Significance between active treatment groups and placebo was tested on raw data analysis.

TABLE 45
Secondary Outcome Analysis - ITT Cohort:
UDI - Did You Experience Lower Abdominal Pressure?
Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 130 | 0.36 | −0.19 | 0.648 | 0.13 | 0.6737 |
| Oxy 6 mg | 119 | 0.42 | −0.23 | 0.718 | 0.09 | 0.6136 |
| Placebo | 133 | 0.50 | −0.32 | 0.724 | | |

*Change = Change in severity of UDI - Did You Experience Lower Abdominal Pressure? (Visit 3 to Visit 7 (or End-of-Treatment)).
**Difference = Difference between active treatment group and placebo.
***P-Value: Significance between active treatment groups and placebo was tested on raw data analysis.

TABLE 46
Secondary Outcome Analysis - ITT Cohort:
UDI - Did You Experience Pain When Urinating?
Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 131 | 0.02 | 0.02 | 0.290 | 0.02 | 0.6638 |
| Oxy 6 mg | 119 | 0.07 | −0.03 | 0.389 | −0.03 | 0.8220 |
| Placebo | 132 | 0.03 | 0.00 | 0.175 | | |

*Change = Change in severity of UDI - Did You Experience Pain When Urinating? (Visit 3 to Visit 7 (or End-of-Treatment)).
**Difference = Difference between active treatment group and placebo.
***P-Value: Significance between active treatment groups and placebo was tested on raw data analysis.

TABLE 47
Secondary outcome Analysis - ITT Cohort:
UDI - Did You Experience Pain in the Lower Abdominal Area or Genital
Area? Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 131 | 0.18 | −0.09 | 0.561 | −0.07 | 0.5503 |
| Oxy 6 mg | 119 | 0.09 | 0.04 | 0.458 | 0.06 | 0.3151 |
| Placebo | 133 | 0.11 | −0.02 | 0.410 | | |

*Change = Change in severity of UDI - Did You Experience Pain in the Lower Abdominal Area or Genital Area? (Visit 3 to Visit 7 (or End-of-Treatment)).
**Difference = Difference between active treatment group and placebo.
***P-Value: Significance between active treatment groups and placebo was tested on raw data analysis.

TABLE 48
Secondary Outcome Analysis - ITT Cohort:
UDI - Did You Experience Heaviness or Dullness in the Pelvic Area?
Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 131 | 0.24 | −0.12 | 0.595 | −0.02 | 0.5703 |
| Oxy 6 mg | 119 | 0.24 | −0.07 | 0.578 | 0.03 | 0.5657 |
| Placebo | 133 | 0.24 | −0.10 | 0.564 | | |

*Change = Change in severity of UDI - Did You Experience Heaviness or Dullness in the Pelvic Area? (Visit 3 to Visit 7 (or End-of-Treatment)).
**Difference = Difference between active treatment group and placebo.
***P-Value: Significance between active treatment groups and placebo was tested on raw data analysis.

TABLE 49
Secondary Outcome Analysis - ITT Cohort:
UDI - Did You Experience a Feeling of Bulging or Protrusion in the
Vaginal Area? Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 131 | 0.13 | −0.05 | 0.398 | −0.07 | 0.0939 |
| Oxy 6 mg | 119 | 0.14 | −0.03 | 0.410 | −0.05 | 0.5616 |
| Placebo | 133 | 0.13 | 0.02 | 0.436 | | |

*Change = Change in severity of UDI - Did You Experience a Feeling of Bulging or Protrusion in the Vaginal Area? (Visit 3 to Visit 7 (or End-of-Treatment)).
**Difference = Difference between active treatment group and placebo.
***P-Value: Significance between active treatment groups and placebo was tested on raw data analysis.

TABLE 50
Secondary Outcome Analysis - ITT Cohort:
UDI - Did You Experience Bulging or Protrusion You Can See in the
Vaginal Area? Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 130 | 0.04 | 0.01 | 0.197 | 0.06 | 0.5211 |
| Oxy 6 mg | 119 | 0.06 | −0.02 | 0.318 | 0.03 | 0.9471 |
| Placebo | 133 | 0.12 | −0.05 | 0.324 | | |

*Change = Change in severity of UDI - Did You Experience Bulging or Protrusion You Can See in the Vaginal Area? (Visit 3 to Visit 7 (or End-of-Treatment)).
**Difference = Difference between active treatment group and placebo.
***P-Value: Significance between active treatment groups and placebo was tested on raw data analysis.

TABLE 51
Secondary Outcome Analysis - ITT Cohort:
UDI - Did You Experience Pelvic Discomfort when Standing or Physically
Exerting Yourself? Change from Baseline (Visit 3) to End-of-Treatment
(Visit 7)

| | | | Mean | Standard | | |
|---|---|---|---|---|---|---|
| Treatments | N | Baseline | Change* | Deviation | Difference** | P-Value*** |
| Oxy 4 mg | 130 | 0.24 | −0.15 | 0.611 | −0.1 | 0.9167 |
| Oxy 6 mg | 119 | 0.10 | −0.01 | 0.460 | 0.04 | 0.6233 |
| Placebo | 133 | 0.13 | −0.05 | 0.377 | | |

*Change = Change in severity of UDI - Did You Experience Pelvic Discomfort when Standing or Physically Exerting Yourself? (Visit 3 to Visit 7 (or End-of-Treatment)).
**Difference = Difference between active treatment group and placebo.
***P-Value: Significance between active treatment groups and placebo was tested on raw data analysis.

TABLE 52
Secondary Outcome Analysis - ITT Cohort:
UDI - Did You Have to Push on the Vaginal Walls to Have a Bowel
Movement? Change from Baseline (Visit 3) to End-of-Treatment (Visit 7)

| | | | Mean | Standard | | |
|---|---|---|---|---|---|---|
| Treatments | N | Baseline | Change* | Deviation | Difference** | P-Value*** |
| Oxy 4 mg | 130 | 0.30 | −0.05 | 0.657 | 0.03 | 0.7222 |
| Oxy 6 mg | 118 | 0.31 | −0.07 | 0.448 | 0.01 | 0.4654 |
| Placebo | 133 | 0.44 | −0.08 | 0.504 | | |

*Change = Change in severity of UDI - Did You Have to Push on the Vaginal Walls to Have a Bowel Movement? (Visit 3 to Visit 7 (or End-of-Treatment)).
**Difference = Difference between active treatment group and placebo.
***P-Value: Significance between active treatment groups and placebo was tested on raw data analysis.

An Incontinence Impact Questionnaire (IIQ) was a list of 30 questions that referred to areas in the patient's life, which may have been influenced or changed by their incontinence problem. The questionnaire measured how severe women found accidental urine loss and/or prolapse had affected their activities, relationships, and feelings. The scale to access how severe the activity/relationship/feeling was affected ranged from 0 to 3, 0 for “not at all”, 1 for “slightly”, 2 for “moderately”, and 3 for “greatly.” In addition, 9 for “not applicable” indicated the environment for recording that scale no longer applied, therefore was treated as missing severity. Analysis results of the change from baseline (Visit 3) to end-of-treatment (Visit 7) for all 30 questions for the ITT cohort are presented below.

For the ITT cohort, statistically significant differences between the treatment group and placebo were found in the assessment of 12 different questions for the mean change from baseline to end-of-study. Both 4 mg/day and 6 mg/day oxybutynin vaginal rings showed a significant reduction in the severity compared to placebo for the affect of incontinence on (1) the patient's ability to travel by car or bus for distances greater than 20 minutes away from home, and (2) sleep. 6 mg/day Oxybutynin vaginal rings were able to achieve or approach statistical significance, compared to placebo, for further reducing the severity of the effect of incontinence on patient's shopping activities, entertainment activities such as going to a movie or concert, ability to travel by car or bus for distances<20 minutes away from home, going places if you are not sure about available restrooms, going on vacation, church or temple attendance, participating in social activities outside your home, frustration, depression, and embarrassment. Tables 53-82 show the analysis of each question in the HQ for the ITT cohort.

TABLE 53
Secondary Outcome Analysis - ITT Cohort:
IIQ - Ability to do Household Chores?
Change from Baseline (Visit 3) to End-of-Treatment (Visit 7)

| Treatments | N | P-Value*** |
|---|---|---|
| Oxy 4 mg | 130 | 0.7651 |
| Oxy 6 mg | 119 | 0.2236 |

***P-Value: Significance between active treatment groups and placebo was tested on raw data analysis.

TABLE 54
Secondary Outcome Analysis - ITT Cohort:
IIQ - Ability to do Usual Maintenance or Repair
Work Done in Home or Yard
Change from Baseline (Visit 3) to End-of-Treatment (Visit 7)

| Treatments | N | P-Value*** |
|---|---|---|
| Oxy 4 mg | 125 | 0.4769 |
| Oxy 6 mg | 112 | 0.3907 |

***P-Value: Significance between active treatment groups and placebo was tested on raw data analysis.

TABLE 55
Secondary Outcome Analysis - ITT Cohort:
IIQ - Shopping Activities
Change from Baseline (Visit 3) to End-of-Treatment (Visit 7)

| Treatments | N | P-Value*** |
|---|---|---|
| Oxy 4 mg | 131 | 0.4450 |
| Oxy 6 mg | 119 | 0.0305 |

***P-Value: Significance between active treatment groups and placebo was tested on raw data analysis.

TABLE 56
Secondary Outcome Analysis - ITT Cohort:
IIQ - Hobbies and Pastime Activities
Change from Baseline (Visit 3) to End-of-Treatment (Visit 7)

| Treatments | N | P-Value*** |
|---|---|---|
| Oxy 4 mg | 129 | 0.3783 |
| Oxy 6 mg | 118 | 0.1616 |

***P-Value: Significance between active treatment groups and placebo was tested on raw data analysis.

TABLE 57
Secondary Outcome Analysis - ITT Cohort:
IIQ - Physical Recreation Activities such as
Walking, Swimming, or Other Exercise
Change from Baseline (Visit 3) to End-of-Treatment (Visit 7)

| Treatments | N | P-Value*** |
|---|---|---|
| Oxy 4 mg | 128 | 0.6786 |
| Oxy 6 mg | 114 | 0.1235 |

P-Value: Significance between active treatment groups and placebo was tested on raw data analysis.

TABLE 58
Secondary Outcome Analysis - ITT Cohort:
IIQ - Entertainment Activities such as Going to
a Movie or Concert?
Change from Baseline (Visit 3) to End-of-Treatment (Visit 7)

| Treatments | N | P-Value*** |
|---|---|---|
| Oxy 4 mg | 128 | 0.3367 |
| Oxy 6 mg | 116 | 0.0326 |

***P-Value: Significance between active treatment groups and placebo was tested on raw data analysis.

TABLE 59
Secondary Outcome Analysis - ITT Cohort:
IIQ - Ability to Travel by Car or Bus for
Distances <20 Minutes Away from Home
Change from Baseline (Visit 3) to End-of-Treatment (Visit 7)

| Treatments | N | P-Value*** |
|---|---|---|
| Oxy 4 mg | 130 | 0.3661 |
| Oxy 6 mg | 119 | 0.0176 |

***P-Value: Significance between active treatment groups and placebo was tested on raw data analysis.

TABLE 60
Secondary Outcome Analysis - ITT Cohort:
IIQ - Ability to Travel by Car or Bus for
Distances >20 Minutes Away from Home
Change from Baseline (Visit 3) to End-of-Treatment (Visit 7)

| Treatments | N | P-Value*** |
|---|---|---|
| Oxy 4 mg | 129 | 0.0159 |
| Oxy 6 mg | 115 | 0.0087 |

***P-Value: Significance between active treatment groups and placebo was tested on raw data analysis.

TABLE 61
Secondary Outcome Analysis - ITT Cohort:
IIQ - Going Places if You Are Not Sure About
Available Restroom?
Change from Baseline (Visit 3) to End-of-Treatment (Visit 7)

| Treatments | N | P-Value*** |
|---|---|---|
| Oxy 4 mg | 129 | 0.1966 |
| Oxy 6 mg | 118 | 0.0009 |

***P-Value: Significance between active treatment groups and placebo was tested on raw data analysis.

TABLE 62
Secondary Outcome Analysis - ITT Cohort:
IIQ - Going on Vacation
Change from Baseline (Visit 3) to End-of-Treatment (Visit 7)

| Treatments | N | P-Value*** |
|---|---|---|
| Oxy 4 mg | 129 | 0.3876 |
| Oxy 6 mg | 115 | 0.0007 |

***P-Value: Significance between active treatment groups and placebo was tested on raw data analysis.

TABLE 63
Secondary Outcome Analysis - ITT Cohort:
IIQ - Church or Temple Attendance
Change from Baseline (Visit 3) to End-of-Treatment (Visit 7)

| Treatments | N | P-Value*** |
|---|---|---|
| Oxy 4 mg | 119 | 0.1848 |
| Oxy 6 mg | 104 | 0.0522 |

***P-Value: Significance between active treatment groups and placebo was tested on raw data analysis.

TABLE 64
Secondary Outcome Analysis - ITT Cohort:
IIQ - Volunteer Activities
Change from Baseline (Visit 3) to End-of-Treatment (Visit 7)

| Treatments | N | P-Value*** |
|---|---|---|
| Oxy 4 mg | 117 | 0.8591 |
| Oxy 6 mg | 104 | 0.1745 |

***P-Value: Significance between active treatment groups and placebo was tested on raw data analysis.

TABLE 65
Secondary Outcome Analysis - ITT Cohort:
IIQ - Employment (Work) Outside the Home
Change from Baseline (Visit 3) to End-of-Treatment (Visit 7)

| Treatments | N | P-Value*** |
|---|---|---|
| Oxy 4 mg | 111 | 0.9359 |
| Oxy 6 mg | 103 | 0.1618 |

***P-Value: Significance between active treatment groups and placebo was tested on raw data analysis.

TABLE 66
Secondary Outcome Analysis - ITT Cohort:
IIQ - Having Friends Visit You in Your Home
Change from Baseline (Visit 3) to End-of-Treatment (Visit 7)

| Treatments | N | P-Value*** |
|---|---|---|
| Oxy 4 mg | 128 | 0.8684 |
| Oxy 6 mg | 116 | 0.2938 |

***P-Value: Significance between active treatment groups and placebo was tested on raw data analysis.

TABLE 67
Secondary Outcome Analysis - ITT Cohort:
IIQ - Participating in Social Activities Outside Your Home
Change from Baseline (Visit 3) to End-of-Treatment (Visit 7)

| Treatments | N | P-Value*** |
|---|---|---|
| Oxy 4 mg | 129 | 0.1091 |
| Oxy 6 mg | 119 | 0.0476 |

***P-Value: Significance between active treatment groups and placebo was tested on raw data analysis.

TABLE 68
Secondary Outcome Analysis - ITT Cohort:
IIQ- Relationship with Friends
Change from Baseline (Visit 3) to End-of-Treatment (Visit 7)

| Treatments | N | P-Value*** |
|---|---|---|
| Oxy 4 mg | 129 | 0.9647 |
| Oxy 6 mg | 118 | 0.1953 |

***P-Value: Significance between active treatment groups and placebo was tested on raw data analysis.

TABLE 69
Secondary Outcome Analysis - ITT Cohort:
IIQ- Relationship with Family Excluding Husband/Companion
Change from Baseline (Visit 3) to End-of-Treatment (Visit 7)

| Treatments | N | P-Value*** |
|---|---|---|
| Oxy 4 mg | 126 | 0.5875 |
| Oxy 6 mg | 118 | 0.0820 |

***P-Value: Significance between active treatment groups and placebo was tested on raw data analysis.

TABLE 70
Secondary Outcome Analysis - ITT Cohort:
IIQ- Ability to Have Sexual Relations
Change from Baseline (Visit 3) to End-of-Treatment (Visit 7)

| Treatments | N | P-Value*** |
|---|---|---|
| Oxy 4 mg | 100 | 0.7603 |
| Oxy 6 mg | 100 | 0.4086 |

***P-Value: Significance between active treatment groups and placebo was tested on raw data analysis.

TABLE 71
Secondary Outcome Analysis - ITT Cohort:
IIQ- Way You Dress
Change from Baseline (Visit 3) to End-of-Treatment (Visit 7)

| Treatments | N | P-Value*** |
|---|---|---|
| Oxy 4 mg | 130 | 0.1192 |
| Oxy 6 mg | 118 | 0.0610 |

***P-Value: Significance between active treatment groups and placebo was tested on raw data analysis.

TABLE 72
Secondary Outcome Analysis - ITT Cohort:
IIQ- Emotional Health
Change from Baseline (Visit 3) to End-of-Treatment (Visit 7)

| Treatments | N | P-Value*** |
|---|---|---|
| Oxy 4 mg | 129 | 0.6604 |
| Oxy 6 mg | 119 | 0.1065 |

***P-Value: Significance between active treatment groups and placebo was tested on raw data analysis.

TABLE 73
Secondary Outcome Analysis - ITT Cohort:
IIQ- Physical Health
Change from Baseline (Visit 3) to End-of-Treatment (Visit 7)

| Treatments | N | P-Value*** |
|---|---|---|
| Oxy 4 mg | 128 | 0.5352 |
| Oxy 6 mg | 118 | 0.1530 |

***P-Value: Significance between active treatment groups and placebo was tested on raw data analysis.

TABLE 74
Secondary Outcome Analysis - ITT Cohort:
IIQ- Sleep
Change from Baseline (Visit 3) to End-of-Treatment (Visit 7)

| Treatments | N | P-Value*** |
|---|---|---|
| Oxy 4 mg | 130 | 0.0177 |
| Oxy 6 mg | 119 | 0.0164 |

***P-Value: Significance between active treatment groups and placebo was tested on raw data analysis.

TABLE 75
Secondary Outcome Analysis - ITT Cohort:
IIQ- Does Fear of Odor Restrict Your Activities?
Change from Baseline (Visit 3) to End-of-Treatment (Visit 7)

| Treatments | N | P-Value*** |
|---|---|---|
| Oxy 4 mg | 128 | 0.3858 |
| Oxy 6 mg | 119 | 0.0873 |

***P-Value: Significance between active treatment groups and placebo was tested on raw data analysis.

TABLE 76
Secondary Outcome Analysis - ITT Cohort:
IIQ- Does Fear of Embarrassment Restrict Your Activities?
Change from Baseline (Visit 3) to End-of-Treatment (Visit 7)

| Treatments | N | P-Value*** |
|---|---|---|
| Oxy 4 mg | 130 | 0.2826 |
| Oxy 6 mg | 118 | 0.4150 |

***P-Value: Significance between active treatment groups and placebo was tested on raw data analysis.

TABLE 77
Secondary Outcome Analysis - ITT Cohort:
IIQ- Nervousness or Anxiety
Change from Baseline (Visit 3) to End-of-Treatment (Visit 7)

| Treatments | N | P-Value*** |
|---|---|---|
| Oxy 4 mg | 129 | 0.7747 |
| Oxy 6 mg | 117 | 0.5468 |

***P-Value: Significance between active treatment groups and placebo was tested on raw data analysis.

TABLE 78
Secondary Outcome Analysis - ITT Cohort:
IIQ- Fear
Change from Baseline (Visit 3) to End-of-Treatment (Visit 7)

| Treatments | N | P-Value*** |
|---|---|---|
| Oxy 4 mg | 126 | 0.8370 |
| Oxy 6 mg | 118 | 0.2871 |

***P-Value: Significance between active treatment groups and placebo was tested on raw data analysis.

TABLE 79
Secondary Outcome Analysis - ITT Cohort:
IIQ- Frustration
Change from Baseline (Visit 3) to End-of-Treatment (Visit 7)

| Treatments | N | P-Value*** |
|---|---|---|
| Oxy 4 mg | 130 | 0.2598 |
| Oxy 6 mg | 119 | 0.0047 |

***P-Value: Significance between active treatment groups and placebo was tested on raw data analysis.

TABLE 80
Secondary Outcome Analysis - ITT Cohort:
IIQ - Anger
Change from Baseline (Visit 3) to End-of-Treatment (Visit 7)

| Treatments | N | P-Value*** |
|---|---|---|
| Oxy 4 mg | 128 | 0.1752 |
| Oxy 6 mg | 118 | 0.6365 |

***P-Value: Significance between active treatment groups and placebo was tested on raw data analysis.

TABLE 81
Secondary Outcome Analysis - ITT Cohort:
IIQ- Depression
Change from Baseline (Visit 3) to End-of-Treatment (Visit 7)

| Treatments | N | P-Value*** |
|---|---|---|
| Oxy 4 mg | 128 | 0.7450 |
| Oxy 6 mg | 118 | 0.0095 |

***P-Value: Significance between active treatment groups and placebo was tested on raw data analysis.

TABLE 82
Secondary Outcome Analysis - ITT Cohort:
IIQ- Embarrassment
Change from Baseline (Visit 3) to End-of-Treatment (Visit 7)

| Treatments | N | P-Value*** |
|---|---|---|
| Oxy 4 mg | 129 | 0.2835 |
| Oxy 6 mg | 119 | 0.0337 |

***P-Value: Significance between active treatment groups and placebo was tested on raw data analysis.

In this double-blind study consisting of a two-week placebo run-in followed by 12 weeks of active treatment or placebo, both the 4 mg/day and the 6 mg/day oxybutynin vaginal rings demonstrated greater reductions compared to placebo from baseline to the end-of-treatment in the weekly total number of reported incontinence episodes and in the number of urge-only incontinence episodes. For the ITT cohort, the 4 mg/day vaginal ring demonstrated a reduction relative to placebo of 2.22 total episodes (p=0.0613) and 2.80 urge-only episodes (p=0.558). The 6 mg/day vaginal ring exhibited a reduction of 2.02 total episodes (p=0.1850) and 2.57 urge-only episodes (p=0.1803) compared to placebo. For the MITT cohort, these reductions were, for the 4 mg/clay oxybutynin vaginal ring, 2.99 total episodes (p=0.0364) and 3.29 urge-only episodes (p=0.544) and, for 6 mg/day vaginal ring, 2.93 total episodes (p=0.0176) and 3.30 urge-only episodes (p=0.0223). The proportions of patients in the 4 mg/day and 6 mg/day oxybutynin vaginal ring groups who reported no incontinence episodes at the end of the treatment was also significantly greater for both the ITT and MITT cohorts.

Urinary frequency was reduced by 0.60 voids per 24 hours for 4 mg/day (p=0.0722) and 0.93 voids per 24 hours for 6 mg/day (p=0.0004) compared to placebo for the ITT cohort. For the MITT cohort, these reductions were 0.70 voids per 24 hours for 4 mg/day (p=0.1039) and 1.0 void per 24 hours for 6 mg/day (p=0.0020). No statistically significant differences between the 4 mg/day and 6 mg/day vaginal rings and placebo were observed with respect to change in average void volume per 24 hours. As a result of a decrease in urinary frequency and no change in average void volume per 24 hours, both active treatment vaginal rings had an average void volume increase of 5.32 mL for the ITT cohort (p=0.0126) and 4.94 mL for the MITT cohort (p=0.0444) compared to placebo.

Mean VAS was reduced by 0.52 for the 4 mg/day (p=0.0199) and 1.23 (p=0.0012) for the 6 mg/day vaginal rings compared to placebo for the ITT cohort. For the MITT cohort, these reductions were 0.44 (p=0.0374) for the 4 mg/day vaginal rings and 1.12 (p=0.0045) for the 6 mg/day vaginal rings.

Results showed that the 4 mg/day vaginal rings provided a level of active treatment effect that exceeded the effect of placebo alone and that the 6 mg/day vaginal rings provided similar results compared to placebo, in addition, was associated with greater reduction in urinary frequency compared to placebo than the 4 mg/day vaginal ring. When considering the MITT cohort consisting of patients who met all three criteria for incontinence at baseline (as opposed to the ITT cohort that included all three patients with analyzable data for the total incontinence episode endpoint), the magnitude of the effect for the oxybutynin vaginal ring groups, especially for the 4 mg/day vaginal rings, was even more evident.

The incidence of treatment-emergent adverse events reported with a frequency of 2% or greater, by body system, is provided in Table 75.

TABLE 75
Treatment-Emergent Adverse Events With an Incidence of 2%
or Greater in Any Treatment Group During Double-Blind
Period - Treated Safety Cohort

| | Placebo | | Oxy 4 Mg | | Oxy 6 Mg | | Total | |
|---|---|---|---|---|---|---|---|---|
| MedDRA System Organ | (N = 155) | | (N = 143) | | (N = 147) | | (N = 143) | |
| Class and Preferred Term | N | % | N | % | N | % | N | % |

INFECTIONS AND INFESTATIONS

| | | | | | | | | |
|---|---|---|---|---|---|---|---|---|
| URINARY TRACT | 7 | 4.52 | 13 | 9.09 | 18 | 12.24 | 38 | 8.54 |
| INFECTION | | | | | | | | |
| SINUSITIS | 2 | 1.29 | 3 | 2.10 | 2 | 1.36 | 7 | 1.57 |
| UPPER RESPIRATORY | 1 | 0.65 | 3 | 2.10 | 1 | 0.68 | 5 | 1.12 |
| TRACT INFECTION | | | | | | | | |
| VULVOVAGINAL | 4 | 2.58 | 3 | 2.10 | 6 | 4.08 | 13 | 2.92 |
| MYCOTIC INFECTION | | | | | | | | |

GASTROINTESTINAL DISORDERS

| | | | | | | | | |
|---|---|---|---|---|---|---|---|---|
| DRY MOUTH | 4 | 2.58 | 7 | 4.90 | 15 | 10.20 | 26 | 5.84 |
| NAUSEA | 1 | 0.65 | 4 | 2.80 | 2 | 1.36 | 7 | 1.57 |
| ABDOMINAL PAIN | 3 | 1.94 | 3 | 2.10 | 3 | 2.04 | 9 | 2.02 |
| CONSTIPATION | 2 | 1.29 | 2 | 1.40 | 4 | 2.72 | 8 | 1.80 |
| DIARRHOEA | 6 | 3.87 | 2 | 1.40 | 5 | 3.40 | 13 | 2.92 |

REPRODUCTIVE SYSTEM AND BREAST DISORDERS

| | | | | | | | | |
|---|---|---|---|---|---|---|---|---|
| VAGINAL | 6 | 3.87 | 5 | 3.50 | 7 | 4.76 | 18 | 4.04 |
| DISCHARGE | | | | | | | | |
| VAGINAL PAIN | 0 | 0.00 | 3 | 2.10 | 0 | 0.00 | 3 | 0.67 |
| VAGINAL | 4 | 2.58 | 2 | 1.40 | 6 | 4.08 | 12 | 2.70 |
| HAEMORRHAGE | | | | | | | | |
| VAGINAL ERYTHEMA | 2 | 1.29 | 0 | 0.00 | 3 | 2.04 | 5 | 1.12 |

MUSCULOSKELETAL AND CONNECTIVE TISSUE DISORDERS

| | | | | | | | | |
|---|---|---|---|---|---|---|---|---|
| BACK PAIN | 4 | 2.58 | 3 | 2.10 | 1 | 0.68 | 8 | 1.80 |

NERVOUS SYSTEM DISORDERS

| | | | | | | | | |
|---|---|---|---|---|---|---|---|---|
| HEADACHE | 2 | 1.29 | 3 | 2.10 | 6 | 4.08 | 11 | 2.47 |

RENAL AND URINARY DISORDERS

| | | | | | | | | |
|---|---|---|---|---|---|---|---|---|
| DYSURIA | 0 | 0.00 | 3 | 2.10 | 2 | 1.36 | 5 | 1.12 |

INVESTIGATIONS

| | | | | | | | | |
|---|---|---|---|---|---|---|---|---|
| HEPATIC ENZYME | 2 | 1.29 | 0 | 0.00 | 3 | 2.04 | 5 | 1.12 |
| INCREASED | | | | | | | | |

The incidence of treatment-emergent adverse events was comparable across treatment groups with the exception of urinary tract infection, dry mouth, and headache. The most commonly reported adverse events were urinary tract infection (12.24% on 6 mg/day oxybutynin vaginal ring, 9.09% on 4 mg/day oxybutynin vaginal ring, and 4.52% on placebo) and dry mouth (10.20% on 6 mg/day oxybutynin vaginal ring, 4.90% on 4 mg/day oxybutynin vaginal ring, and 2.58% on placebo); both adverse events were associated with incidence rates that increased with dose. The incidence rates of dry mouth compare favorably to the 29-61% rate reported for an oral, extended release formulation of oxybutynin (Ditropan XL®) and are similar to the rates of 4.9-9.6% seen in a twice weekly transdermal oxybutynin product. All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. An intravaginal device comprising:
(a) an annular first matrix comprising a pocket and a pocket wall, wherein the pocket wall has a uniform thickness, and wherein the pocket wall encompasses the pocket; and
(b) a second matrix comprising an anticholinergic agent, wherein the second matrix is located in the pocket.

2. The intravaginal device of claim 1, wherein the first matrix comprises an optionally substituted polymer selected from the group consisting of polysiloxane polymers, polyalkylene polymers, polystyrene polymers, polyvinyl acetate polymers, polyvinyl chloride polymers, polyester polymers, polyurethane polymers, acrylic polymers, nylon polymers, dacron polymers, teflon polymers, and combinations thereof.

3. The intravaginal device of claim 2, wherein the optionally substituted polymer is a polysiloxane polymer of Formula (I): 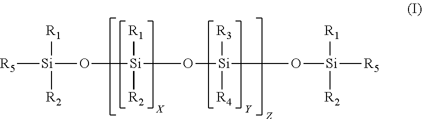
wherein
X is 1 to 200;
Y is 1 to 200;
Z is 1 to 300; and
R₁, R₂, R₃, R₄, and R₅ are independently selected from the group consisting of (C₁₋₆)alkyl, amino(C₁₋₆)alkyl, hydroxy(C₁₋₆)alkyl, haloalkyl, cyano(C₁₋₆)alkyl, thio(C₁₋₆)alkyl, carboxy(C₁₋₆)alkyl, aryl(C₁₋₆)alkyl, (C₁₋₆)alkoxy(C₁₋₆)alkyl, (C₂₋₆)alkenyl, amino(C₃₋₁₀)alkenyl, hydroxy(C₃₋₁₀)alkenyl, halo(C₂₋₆)alkenyl, cyano(C₂₋₆)alkenyl, thio(C₃₋₁₀)alkenyl, carboxy(C₃₋₁₀)alkenyl, aryl(C₂₋₆)alkenyl, (C₂₋₆)alkynyl, (C₁₋₆)heteroalkyl, (C₂₋₆)heteroalkenyl, (C₂₋₆)heteroalkynyl, (C₁₋₆)alkoxy, (C₃₋₁₀)alkenyloxy, (C₁₋₆)alkylenedioxy, amino(C₂₋₆)alkoxy, hydroxy(C₂₋₆)alkoxy, halo(C₁₋₆)alkoxy, cyano(C₁₋₆)alkoxy, thio(C₁₋₆)alkoxy, carboxy(C₂₋₆)alkoxy, aryl(C₁₋₆)alkoxy, (C₁₋₆)alkoxy(C₂₋₆)alkoxy, halo(C₁₋₆)alkoxy(C₂₋₆)alkoxy, mono(C₁₋₆)alkylamino, di(C₁₋₆)alkylamino, (C₁₋₆)alkylcarbonylamino, (C₂₋₆)alkenylcarbonylamino, (C₆₋₁₄)arylcarbonylamino, (C₁₋₆)alkoxycarbonylamino, (C₆₋₁₀)aryloxycarbonylamino, (C₁₋₆)alkylcarbonyl, (C₂₋₆)alkenylcarbonyl, (C₆₋₁₀)arylcarbonyl, (C₁₋₆)alkoxycarbonyl, (C₆₋₁₄)aryloxycarbonyl, (C₁₋₆)alkylsulfonylamino, (C₂₋₆)alkenylsulfonylamino, and (C₆₋₁₄)arylsulfonylamino.

4. The intravaginal device of claim 3, wherein at least one of R₁, R₂, R₃, and R₄ is a haloalkyl.

5. The intravaginal device of claim 3, wherein
X is 1 to 2;
Y is 1 to 2;
Z is 100 to 200; and
R₁ is trifluoropropyl;
R₂, R₃, and R₄ are independently C₁-C₃ alkyl; and
R₅ is vinyl.

6. The intravaginal device of claim 3, wherein the optionally substituted polymer is 3,3,3-trifluoropropyl methyldimethyl polysiloxane.

7. The intravaginal device of claim 4, wherein the first matrix comprises 50% to 100% by weight halogenated siloxane polymer.

8. (canceled)

9. The intravaginal device of claim 1, wherein the first matrix comprises 80% to 95% by weight of the device.

10. (canceled)

11. The intravaginal device of claim 1, wherein the pocket extends from 80° to 120° around the perimeter of the first matrix.

12. The intravaginal device of claim 1, wherein the pocket has a cross-sectional diameter of 3 mm to 8 mm.

13. The intravaginal device of claim 1, wherein the pocket wall has a uniform thickness of 1 mm to 4 mm.

14. The intravaginal device of claim 1, wherein the pocket has a volume of 0.7 cm³ to 1.5 cm³.

15. The intravaginal device of claim 1, wherein the second matrix comprises an optionally substituted polymer selected from the group consisting of polysiloxane polymers, polyalkylene polymers, polystyrene polymers, polyvinyl acetate polymers, polyvinyl chloride polymers, polyester polymers, polyurethane polymers, acrylic polymers, nylon polymers, dacron polymers, teflon polymers, and combinations thereof.

16. The intravaginal device of claim 15, wherein the second matrix comprises a polysiloxane polymer.

17. The intravaginal device of claim 16, wherein the second matrix comprises a polysiloxane polymer of Formula (II): 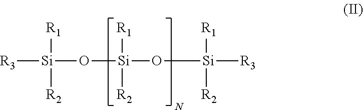
wherein
R₁, R₂, and R₃ are independently selected from the group consisting of alkoxy, alkyl, alkynyl, alkynyl, alkenyl, alkylacryloyloxy, acryloyloxy, alkenylalkyl, aryl, and hydrogen; and
N is 50 to 300.

18. The intravaginal device of claim 17, wherein R₁ and R₂ are independently alkyl or hydrogen.

19. The intravaginal device of claim 15, wherein the second matrix comprises 30% to 80% by weight polysiloxane polymer.

20. (canceled)

21. The intravaginal device of claim 1, wherein the second matrix comprises 5% to 50% by weight of the device.

22. The intravaginal device of claim 1, wherein the anticholinergic agent is homogenously dispersed throughout the second matrix.

23. The intravaginal device of claim 1, wherein the anticholinergic agent is selected from the group consisting of oxybutynin, tolterodine, trospium, solifenacin, darifenacin, dicyclomine, propantheline, propiverine, bethanechol, methylbenactyzium, scopolamine, and pharmaceutically acceptable salts thereof.

24. The intravaginal device of claim 23, wherein the anticholinergic agent is oxybutynin or a pharmaceutically acceptable salt thereof.

25. The intravaginal device of claim 1, wherein the anticholinergic agent comprises 20% to 70% by weight of the second matrix.

26. The intravaginal device of claim 1, wherein the first matrix further comprises a slit, wherein the slit extends a length of the pocket.

27. A method of making an intravaginal device, the method comprising:
(a) placing a first matrix into a mold, the mold being shaped so as to form an annular intravaginal device comprising a pocket and a pocket wall, wherein the pocket wall has a uniform thickness, and wherein the pocket wall encompasses the pocket;
(b) curing the first matrix;
(c) placing a second matrix comprising an anticholinergic agent in the pocket; and
(d) curing the second matrix.

28. The method of claim 27, wherein the mold is shaped so as to form an annular intravaginal device comprising a pocket and a pocket wall, wherein the pocket wall has a uniform thickness, wherein the pocket wall encompasses the pocket, and wherein a slit extends a length of the pocket.

29. (canceled)

30. (canceled)

31. The method of claim 27, wherein the anticholinergic agent is oxybutynin or a pharmaceutically acceptable salt thereof.
